(19) United States
(12) Patent Application Publication
(10) Pub. No.: US 2002/0177800 A1
(43) Pub. Date: Nov. 28, 2002

(54) Aspiration catheters and method of use

(76) Inventors: Celso J. Bagaoisan, Union City, CA (US); Hung V. Ha, San Jose, CA (US); Mukund R. Patel, San Jose, CA (US); Sivette Lam, San Jose, CA (US); Ketan P. Muni, San Jose, CA (US); Gholam-Reza Zadno-Azizi, Freemont, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/125,180

(22) Filed: Apr. 16, 2002

Related U.S. Application Data
(60) Provisional application No. 60/284,287, filed on Apr. 16, 2001.

(51) Int. Cl. A61M 37/00; A61M 1/00; A61M 5/19

(52) U.S. Cl. 604/6.12; 604/35; 604/131; 604/164.13

(57) ABSTRACT

Various methods and apparatus are provided for aspirating emboli and other particles from the vasculature of a patient, particularly within saphenous vein grafts, coronary arteries, carotid arteries and similar vessels. One embodiment of an aspiration catheter is particularly well suited for delivery over a guidewire. Preferably, the guidewire is hollow and carries a distal occlusive device, and has a low profile to facilitate passage into small vessels. The aspiration catheter comprises an elongate body having a guidewire lumen positioned within an aspiration lumen, thereby providing a low profile catheter having a round cross-sectional shape. The aspiration lumen has an angled aspiration mouth which improves evacuation efficiency, and facilitates aspiration of larger particles within vessels. The angle of the aspiration mouth prevents suction between the mouth and the occlusive device, thereby reducing forced movement of the occlusive device while it is deployed during aspiration procedures.

### RELATED APPLICATION

This application is claims the benefit of U.S. Provisional Application No. 60/284,287, entitled ASPIRATION CATHETERS AND METHOD OF USE, filed Apr. 16, 2001, the entirety of which is hereby incorporated by reference herein.

### BACKGROUND OF THE INVENTION

1. Field of the Invention

The present invention relates to aspiration catheters for aspirating emboli, thrombi, and other types of particles from the vasculature of a patient, the apparatus being particularly well suited for aspiration within saphenous vein grafts, coronary arteries, carotid arteries and similar vessels.

2. Description of the Related Art

Human blood vessels often become occluded or completely blocked by plaque, thrombi, other deposits, emboli or other substances, which reduce the blood carrying capacity of the vessel. Should the blockage occur at a critical place in the circulatory system, serious and permanent injury, or even death, can occur. To prevent this, some form of medical intervention is usually performed when significant occlusion is detected.

Coronary heart disease is an extremely common disorder in developed countries, and is the leading cause of death in the U.S. Damage to or malfunction of the heart is caused by narrowing or blockage of the coronary arteries (atherosclerosis) that supply blood to the heart. The coronary arteries are first narrowed and may eventually be completely blocked by plaque, and may further be complicated by the formation of thrombi (blood clots) on the roughened surfaces of the plaques. Myocardial infarction can result from atherosclerosis, especially from an occlusive or near occlusive thrombi overlying or adjacent to the atherosclerotic plaque, leading to death of portions of the heart muscle. Thrombi and emboli also often result from myocardial infarction, and these clots can block the coronary arteries, or can migrate further downstream, causing additional complications.

Various types of intervention techniques have been developed which facilitate the reduction or removal of the blockage in the blood vessel, allowing increased blood flow through the vessel. One technique for treating stenosis or occlusion of a blood vessel is balloon angioplasty. A balloon catheter is inserted into the narrowed or blocked area, and the balloon is inflated to expand the constricted area. In many cases, near normal blood flow is restored. It can be difficult, however, to treat plaque deposits and thrombi in the coronary arteries, because the coronary arteries are small, which makes accessing them with commonly used catheters difficult.

Other types of intervention include atherectomy, deployment of stents, introduction of specific medication by infusion, and bypass surgery. Each of these methods are not without the risk of embolism caused by the dislodgement of the blocking material which then moves downstream. In addition, the size of the blocked vessel may limit percutaneous access to the vessel.

In coronary bypass surgery, a more costly and invasive form of intervention, a section of a vein, usually the saphenous vein taken from the leg, is used to form a connection between the aorta and the coronary artery distal to the obstruction. Over time, however, the saphenous vein graft may itself become diseased, stenosed, or occluded, similar to the bypassed vessel. Atherosclerotic plaque in saphenous vein grafts tends to be more friable and less fibrocalcific than its counterpart in native coronary arteries.

Diffusely diseased old saphenous vein grafts with friable atherosclerotic lesions and thrombi have therefore been associated with iatrogenic distal embolic debris. Balloon dilatation of saphenous vein grafts is more likely to produce symptomatic embolization than dilatation of the coronary arteries, not only because of the difference in the plaque but also because vein grafts and their atheromatous plaques are generally larger than the coronary arteries to which they are anastomosed. Once the plaque and thrombi are dislodged from the vein, they can move downstream, completely blocking another portion of the coronary artery and causing myocardial infarction. In fact, coronary embolization as a complication of balloon angioplasty of saphenous vein grafts is higher than that in balloon angioplasty of native coronary arteries. Therefore, balloon angioplasty of vein grafts is performed with the realization that involvement by friable atherosclerosis is likely and that atheroembolization represents a significant risk.

Because of these complications and high recurrence rates, old diffusely diseased saphenous vein grafts have been considered contraindications for angioplasty and atherectomy, severely limiting the options for minimally invasive treatment. However, some diffusely diseased or occluded saphenous vein grafts may be associated with acute ischemic syndromes, necessitating some form of intervention.

There is therefore a need for improved methods of treatment for occluded vessels such as saphenous vein grafts and the smaller coronary arteries which decrease the risks to the patient.

### SUMMARY OF THE INVENTION

Various methods and apparatus are provided for aspirating emboli and other particles from the vasculature of a patient, particularly within saphenous vein grafts, coronary arteries, carotid arteries and similar vessels. One embodiment of an aspiration catheter is particularly well suited for delivery over a guidewire. Preferably, the guidewire is hollow and carries a distal occlusive device, and has a low profile to facilitate passage into small vessels. The aspiration catheter comprises an elongate body having a guidewire lumen positioned within an aspiration lumen, thereby providing a low profile catheter having a round cross-sectional shape. The aspiration lumen has an angled aspiration mouth which improves evacuation efficiency, and facilitates aspiration of larger particles within vessels. The angle of the aspiration mouth prevents suction between the mouth and the occlusive device, thereby reducing forced movement of the occlusive device while it is deployed during aspiration procedures.

In one embodiment, an aspiration catheter is provided for removing emboli or other particles from a blood vessel. The aspiration catheter comprises a first elongate body and a second elongate body. The first elongate body has a proximal end and a distal end and a first lumen extending therethrough. The second elongate body has a proximal end and a distal end and a second lumen and a third lumen extending therethrough. The first lumen of the first elongate body is inserted into the second lumen of the second elongate body to form an aspiration lumen extending from the proximal end of the first lumen to the distal end of the second lumen. The first lumen is secured to the second lumen by a length of shrink tubing which is contracted around an interface between the first and second lumens. The third lumen extends within the second lumen and is substantially parallel to the second lumen. The third lumen has a proximal end proximal to the distal end of the first lumen and distal to the proximal end of the second lumen, and a distal end distal to the distal end of the second lumen. The third lumen is adapted to receive a guidewire therethrough. The proximal end of the first elongate body is fitted with an aspiration port in fluid communication with the second lumen. The distal end of the second lumen comprises an aspiration mouth having an oblique angle relative to a longitudinal axis of the second lumen. The aspiration mouth faces away from the third lumen and is in fluid communication with the second lumen.

In another embodiment, a method of fabricating an aspiration catheter for removing emboli or other particles from a blood vessel is provided. A first elongate tubular body having a single lumen extending therethrough is provided. An aspiration port is affixed to a proximal end of the first elongate tubular body such that the aspiration port is in fluid communication with the single lumen. A distal portion of the first elongate tubular body is heated and stretched to narrow the diameter of the distal portion. Material is removed from one side of a distal end of the first elongate tubular body to form a cut section. A second elongate tubular body is provided. The second elongate tubular body has a primary lumen extending therethrough and a secondary lumen extending within the primary such that the primary lumen has a crescent cross-section and the second elongate tubular body has a round cross-section. The secondary lumen is substantially parallel with the primary lumen and extends distally beyond an aspiration mouth of the primary lumen, and forms a distal end of the aspiration catheter. The aspiration mouth has an oblique angle relative to a longitudinal axis of the primary lumen and is in fluid communication therewith. The aspiration mouth faces away from the secondary lumen. A rod is inserted into the distal end of the secondary lumen such that a distal end of the rod is outside of the distal end of the secondary lumen and a proximal end of the rod protrudes from a proximal end of the secondary lumen. A junction is formed by inserting the distal end of the first elongate tubular body into a proximal end of the primary lumen such that the cut section faces towards the secondary lumen. A length of shrink tubing is positioned over the junction and caused to contract thereon. A marker is inserted within the distal end of the secondary lumen, and positioned within the secondary lumen at the position of the aspiration mouth.

In another embodiment, an aspiration catheter is provided for removing emboli or other particles from a blood vessel. The aspiration catheter comprises a proximal portion having a first lumen extending therethrough and a distal portion having a second lumen and a third lumen extending therethrough. The third lumen extends within the second lumen and is substantially parallel therewith such that the second lumen has a crescent cross-section and the distal portion has a round cross-section. A distal end of the second lumen comprises an aspiration mouth having an oblique angle relative to a longitudinal axis of the second lumen. The aspiration mouth faces away from the third lumen and is in fluid communication with the second lumen. The third lumen is sized and configured to receive a guidewire. A proximal end of the proximal portion is fitted with an aspiration port in fluid communication with the second lumen. The aspiration port is configured to receive a source of negative pressure.

The aspiration catheter further comprises a junction, which comprises the proximal portion being distally inserted into a proximal end of the distal portion such that the first lumen is in fluid communication with the second lumen. A distal end of the proximal portion comprises a cut section extending proximally from the distal end. The cut section has a length directly proportional to a length of the proximal portion which is inserted into the distal portion. The distal portion comprises a cut section extending distally from a proximal end of the second lumen to a distance proximal of the proximal end of the third lumen. The proximal portion is secured to the distal portion by a length of shrink tubing which is contracted around the junction.

Another embodiment provides an aspiration catheter for removing emboli or other particles from a blood vessel. The aspiration catheter comprises a dual lumen portion, a single lumen portion and an aspiration port. The dual lumen portion has a primary lumen and a secondary lumen. The primary lumen has a distal aspiration mouth in fluid communication with the primary lumen, and the secondary lumen extends within the primary lumen and protruding distally beyond the aspiration mouth to form a distal end of the aspiration catheter. The secondary lumen is substantially parallel with the primary lumen such that the primary lumen has a crescent cross-section and the dual lumen portion has a round cross-section. The secondary lumen is sized and configured to receive a standard-size coronary guidewire. The aspiration mouth defines an oblique opening facing away from the secondary lumen. The single lumen portion has a distal end inserted into a proximal end of the primary lumen such that a proximal end of the single lumen portion is in fluid communication with the aspiration mouth. The aspiration port is disposed on the proximal end of the single lumen portion and is in fluid communication with the aspiration mouth. The distal end of the single lumen portion comprises a cut section extending proximally from the distal end, the cut section having a length which is directly proportional to a length of the single lumen portion which is inserted into the dual lumen portion. The single lumen portion is secured to the dual lumen portion by a length of shrink tubing which is contracted around an interface between the single lumen portion and the dual lumen portion.

Still another embodiment provides a method of fabricating an aspiration catheter for removing emboli or other particles from a blood vessel. A first elongate tubular body is provided. The first elongate tubular body has a single lumen extending from a distal end to a proximal end. The proximal end is fitted with an aspiration port in fluid communication with the single lumen, and the distal end has an oblique angle relative to a longitudinal axis of the first elongate tubular body. The distal end has a cut section extending proximally on one side. A second elongate tubular body is provided. The second elongate tubular body has a primary lumen extending therethrough and a secondary lumen extending within the primary lumen such that the primary lumen has a crescent cross-section and the second elongate tubular body has a round cross-section. The secondary lumen is substantially parallel with the primary lumen. The distal end of the first elongate tubular body is inserted into the proximal end of the primary lumen with the cut section facing towards the secondary lumen. The distal end of the first elongate tubular body is secured to the proximal end of the primary lumen.

Another embodiment provides an aspiration catheter for removing emboli or other particles from a blood vessel. The aspiration catheter comprises a shaft which comprises a distal end and a proximal end and has at least a first lumen and a second lumen extending therebetween. The second lumen extends within the first lumen such that the first lumen has a crescent cross-section and the shaft has a round cross-section. An aspiration port is disposed on the proximal end and is in fluid communication with the first lumen. An aspiration mouth is disposed on the distal end and is in fluid communication with the first lumen. The aspiration mouth defines an oblique opening which faces away from the second lumen. An opening is disposed between the distal end and the proximal end of the shaft. The opening defines a proximal end of the second lumen and is in fluid communication with a distal end of the second lumen.

### BRIEF DESCRIPTION OF THE DRAWINGS

FIG. 1 is a perspective view of an integrated inflation/deflation device, shown operably coupled to an illustrative inflation adapter and a balloon catheter deployed in a blood vessel.

FIG. 2A is a side view of a balloon catheter which can be used with one preferred embodiment of an aspiration catheter.

FIG. 2B is a longitudinal cross-sectional view of the distal end of the balloon catheter of FIG. 2A.

FIG. 2C is an enlarged cross-sectional view of the proximal end of the balloon of FIG. 2B.

FIG. 3 shows the inflation adapter of FIG. 1 having a low profile catheter valve and balloon catheter placed therewithin.

FIG. 4A is a partial cross-sectional view of a low profile catheter valve.

FIG. 4B is an enlarged view of the low profile catheter valve of FIG. 4A, showing the valve in an open position (and a closed position shown in phantom).

FIG. 5 is a side view of an illustrative single-operator type aspiration catheter.

FIG. 6 is a side view of an over-the-wire aspiration catheter.

FIG. 7 is a cross sectional view of the aspiration catheter of FIG. 6, taken along line 7-7 in FIG. 6.

FIG. 8 is a cross sectional view of the aspiration catheter of FIG. 7, taken along line 7-7, showing a guidewire over which the aspiration catheter rides.

FIG. 9 is a side view of a single-operator type aspiration catheter.

FIG. 10 is a cross sectional view of a proximal section of the aspiration catheter of FIG. 9, taken along line 10-10 of FIG. 9.

FIG. 11A is a cross sectional view of one embodiment of a distal section of the aspiration catheter of FIG. 9, taken along line 11-11 of FIG. 9.

FIG. 11B is a cross sectional view of another embodiment of a distal end of the aspiration catheter of FIG. 9, also taken along line 11-11 of FIG. 9, showing a slit in the outer wall of a guidewire lumen through which a guidewire can be inserted and removed.

FIG. 12 is a side view of another embodiment of an over-the-wire aspiration catheter.

FIG. 13 is a cross-sectional view of the aspiration catheter of FIG. 12, taken along line 13-13.

FIGS. 14A-14C are side views illustrating various embodiments of the distal end of an aspiration catheter.

FIG. 15 is a side view of another embodiment of a single-operator type aspiration catheter.

FIG. 16 is a cross-sectional view of the aspiration catheter of FIG. 15, taken along line 16-16.

FIG. 17 is a side view of another embodiment of an aspiration catheter.

FIGS. 18A-18D are cross-sectional views of the aspiration catheter shown in FIG. 17.

FIG. 19 is a cross-sectional view of a junction of proximal and distal sections of the catheter shown in FIG. 17, showing bonding of a single-lumen portion and a dual-lumen portion.

FIG. 20 is a cross-sectional view of the catheter of FIG. 19, taken through line 20-20.

FIG. 21 is a side view of another embodiment of an aspiration catheter in which a guidewire lumen is internal to an aspiration lumen.

FIG. 21A is a side cut-away view of a dual lumen tubing of the aspiration catheter of FIG. 21.

FIGS. 22-24 are cross-sectional views of the catheter shown in FIG. 21.

FIG. 25 is a cross-sectional view of a junction of a proximal section and a distal section of the catheter shown in FIG. 21, showing a bonding of a single lumen portion and a dual lumen portion.

FIG. 26 is a cross-sectional view of the aspiration catheter of FIG. 21 having an ultrasound sensor.

FIG. 27 is a side cut away view of a guidewire inserted into a saphenous vein graft, wherein the guidewire has a radiopaque marker for targeting by an external shock wave generator.

### DETAILED DESCRIPTION OF THE PREFERRED EMBODIMENT

Preferred embodiments described below relate particularly to aspiration catheters for aspirating emboli and other types of particles from the vasculature of a patient. Although these embodiments describe certain types of aspiration catheters and methods of use, it will be appreciated that the description is illustrative only and should not to be construed as limiting in any way; and thus, other structures and configurations may be used. Furthermore, various applications and modifications of the embodiments described herein, which may occur to those skilled in the art, are also encompassed by the general concepts described below.

### I. Overview of Occlusion System

A. Balloon System

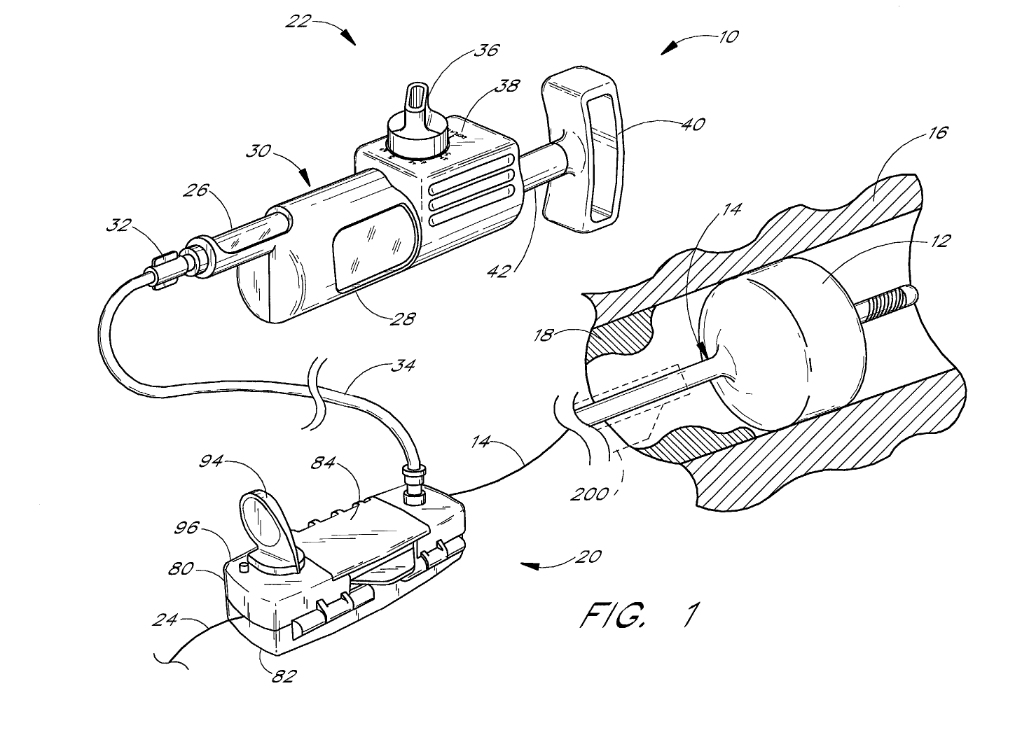

FIG. 1 illustrates generally the components of one exemplifying occlusion balloon guidewire system 10. As described in further detail below, an occlusion balloon 12 used in this system is delivered on a guidewire 14 to a location in a blood vessel 16 distal of an occlusion 18. Through the use of an adapter 20 and an inflation/deflation device or syringe assembly 22, the balloon 12 is inflated through a lumen in the guidewire 14 to occlude the vessel 16 distal to the occlusion 18. Through the use of a valve 24 described below, the adapter 20 can be removed from the proximal end of the guidewire 14 while the balloon 12 remains inflated. With the proximal end of the guidewire 14 free of obstructions, other catheters can be delivered and exchanged over the guidewire 14 to perform therapy and treatment on the occlusion 18. Because the balloon 12 on the guidewire 14 remains inflated distal to the occlusion 18, any particles broken off during treatment of the occlusion 18 are isolated proximal to the balloon 12. These particles can be removed using an aspiration catheter 200 (shown in phantom in FIG. 1) delivered over the guidewire 14. After the particles are removed, the adapter 20 and the inflation/deflation device 22 can be reattached to the proximal end of the guidewire 14 to deflate the balloon 12.

It is to be understood that “occlusion” as used herein with reference to a blood vessel includes both complete and partial occlusions, stenoses, emboli, thrombi, plaque and any other substance which at least partially occludes the lumen of the blood vessel. The term “occlusive device” as used herein includes balloons, filters and other devices which are used to partially or completely occlude the blood vessel prior to performing therapy on the occlusion. The methods described herein are particularly suited for use in removal of occlusions from saphenous vein grafts, coronary and carotid arteries, and vessels having similar pressures and flow.

1. Syringe Assembly

The preferred embodiments described herein may comprise or be used in conjunction with a syringe assembly as described in U.S. patent application Ser. No. 09/338,375, filed Jun. 23, 1999, entitled INTEGRATED INFLATION/DEFLATION DEVICE AND METHOD, the disclosure of which is incorporated herein by reference in its entirety.

One preferred embodiment of a syringe assembly 22 for inflation and deflation of an occlusion balloon is shown in FIG. 1. The syringe assembly 22 comprises a low-volume inflation syringe 26 and a high capacity or reservoir syringe 28 encased together in a housing 30. The syringe assembly 22 is preferably attached via a connector 32 and a short tube 34 to an adapter 20 within which a low profile catheter valve 24 and a balloon catheter 14 are engaged during use. The balloon catheter 14 is shown in an inflated state within a blood vessel 16 in FIG. 1. An inflation/deflation knob 36 is disposed on the outside of the housing 30. Indicia 38 are preferably located on the housing 30 adjacent the knob 36 so that a physician using the device can monitor the precise volume of liquid delivered by the inflation syringe 22. As depicted, the indicia 38 preferably comprise numbers corresponding to the size and shape of the balloon 12 used. When the knob 36 is rotated from the “DEFLATE” or “0” position to the number corresponding to the balloon 12 in use, the syringe assembly 22 delivers the fluid volume associated with that balloon size. Alternatively, the indicia 38 could indicate the standard or metric volume of fluid delivered at each position. A handle 40 is disposed on a proximal end of the plunger 42. Preferably, the handle 40 is large, as illustrated in FIG. 1, and is easily held in a physician's hand.

2. Occlusion Balloon Guidewire

The occlusion balloon guidewire system generally illustrated in FIG. 1 performs the function of occluding a vessel and allowing for the slidable insertion or advancement of various catheters and other devices. The term “catheter” as used herein is therefore intended to include both guidewires and catheters with these desired characteristics. The term “occlusion” refers to both partial and total occlusion of a vessel as mentioned above.

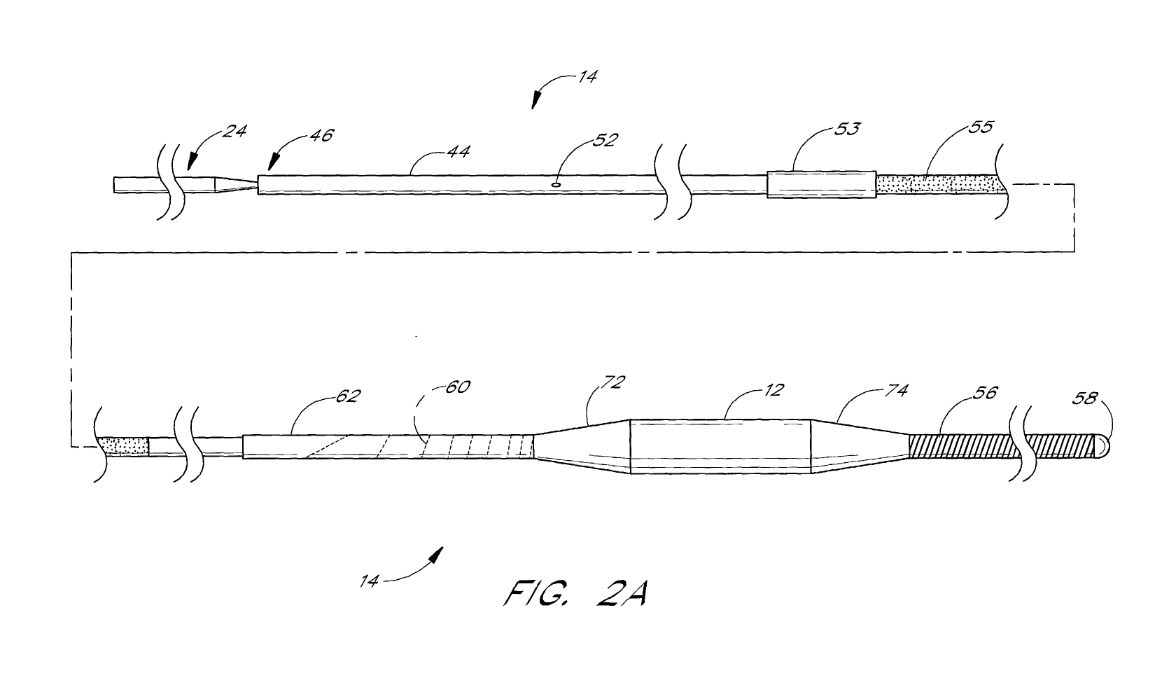

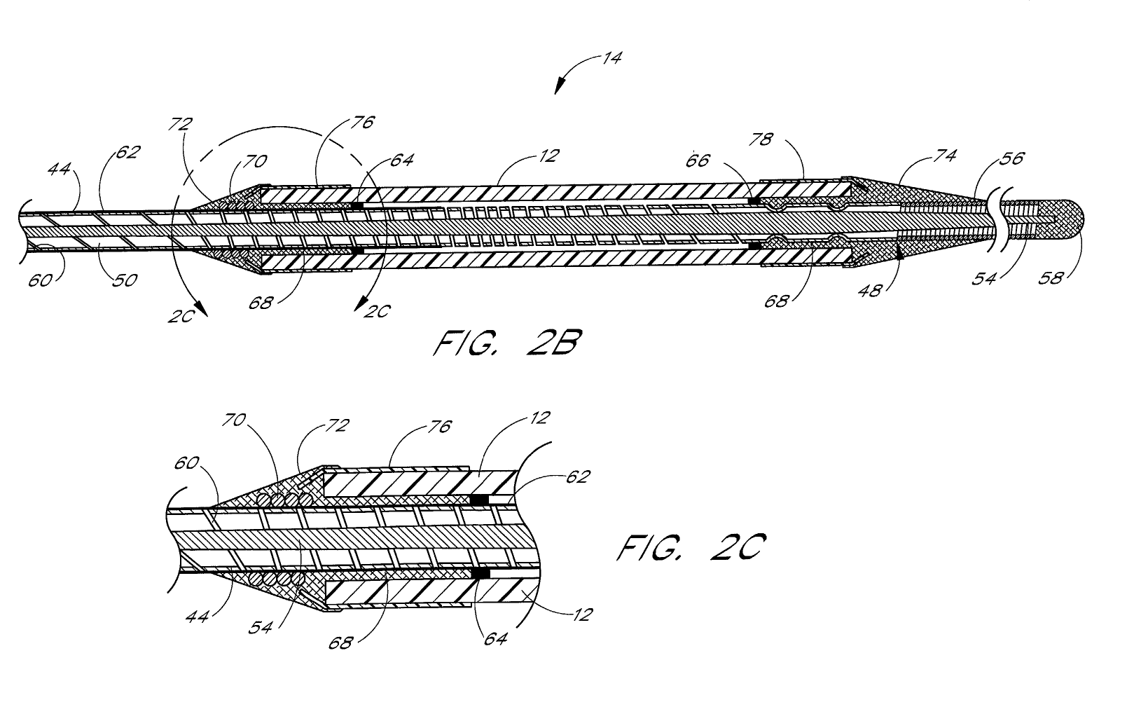

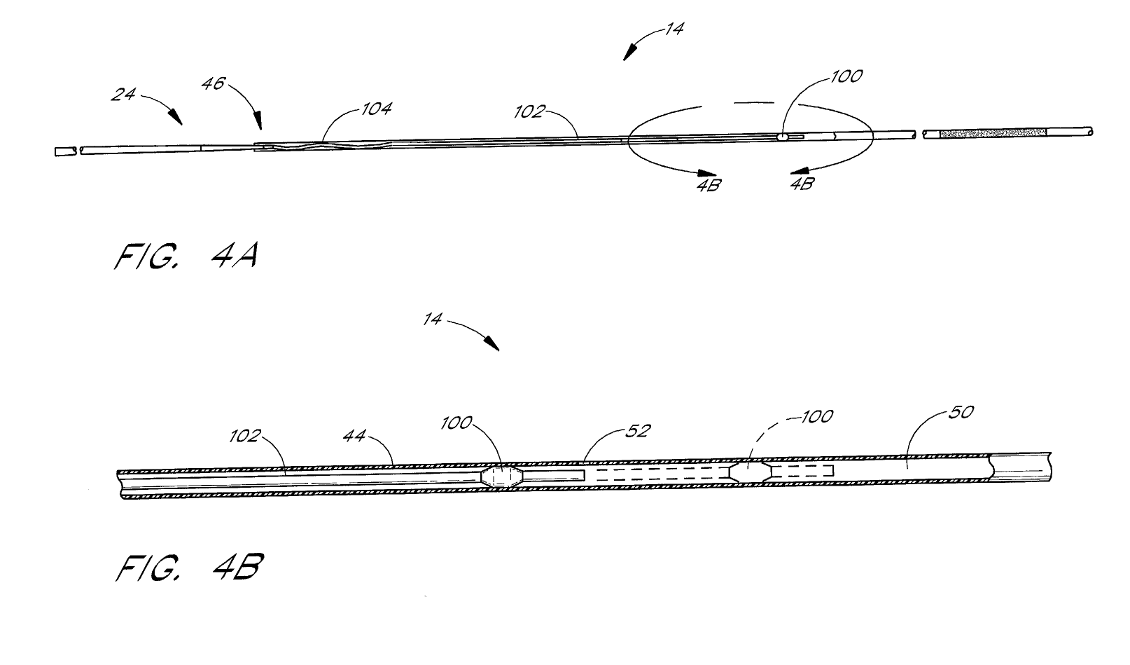

As shown in FIG. 2A, a balloon guidewire catheter 14 generally comprises an elongate flexible tubular body 44 extending between a proximal control end 46, corresponding to a proximal section of the tubular body 44, and a distal functional end 48 (FIG. 2B), corresponding to a distal section of tubular body 44. The tubular body 44 has a central lumen 50, which extends between the proximal and distal ends 46, 48. An inflation port 52, shown also in FIGS. 4A and 4B described below, is provided on the tubular body 44 near the proximal end 46. The inflation port 52 is in fluid communication with lumen 50 such that fluid passing through the inflation port 52 into or out of the lumen 50 may be used to inflate or deflate an inflatable balloon 12 in communication with the lumen 50.

A valve 24, as described below, is inserted into the proximal end 46 of the tubular body 44 to control inflation of the balloon 12, mounted on the distal end of the tubular body 44 through the inflation port 52. The inflation port 52 is preferably formed by electric discharge machining (EDM). A proximal marker 53, which is preferably made of gold, is placed over the tubular body 44 distal to the inflation port 52. Distal to the marker 53, a nonuniform coating 55 of polymer material, more preferably polytetrafluoroethylene (TFE), is applied to the tubular body 44, terminating proximal to a shrink tubing 62. The shrink tubing 62 extends up to and within the balloon 12, as described below. Adhesive tapers 72 and 74 extend from the proximal and distal ends of the balloon 12, respectively. The proximal taper 72 preferably extends from the proximal end of the balloon to the shrink tubing 62 on the tubular body 44, while the distal taper 74 extends to coils 56 extending from the distal end 48 (FIG. 2B) of the tubular body 44. The coils 56 terminate in a distal ball 58.

The length of the tubular body 44 may be varied considerably depending on the desired application. For example, when catheter 14 serves as a guidewire for other catheters in a conventional percutaneous transluminal coronary angioplasty procedure involving femoral artery access, tubular body 44 is comprised of a hollow hypotube having a length ranging from about 160 centimeters to about 320 centimeters, with a length of about 180 centimeters being optimal for a single-operator device, or 300 centimeters for over-the-wire applications. Alternatively, for different treatment procedures not requiring as long a length of the tubular body 44, shorter lengths of the tubular body 44 may be provided.

The tubular body 44 generally has a circular cross-sectional configuration with an outer diameter within the range from about 0.008 inches to about 0.14 inches. In applications where the catheter 14 is to be used as a guidewire for other catheters, the outer diameter of tubular body 44 ranges from about 0.010 inches to about 0.038 inches and preferably is about 0.014 to about 0.020 inches in outer diameter or smaller. Noncircular cross-sectional configurations of the lumen 50 can also be adapted for use with the catheter 14. For example, triangular, rectangular, oval and other noncircular cross-sectional configurations are also easily incorporated for use with the catheter 14, as will be appreciated by those of skill in the art. The tubular body 44 may also have variable cross-sectional configurations.

The tubular body 44 has sufficient structural integrity or “pushability” to permit the catheter 14 to be advanced through the vasculature of a patient to distal arterial locations without buckling or undesirable kinking of the tubular body 44. It is also desirable for the tubular body 44 to have the ability to transmit torque such as in those embodiments wherein it may be desirable to rotate the tubular body 44 after insertion into a patient. A variety of biocompatible materials known by those of skill in the art to possess these properties and to be suitable for catheter manufacture may be used to produce the tubular body 44. For example, the tubular body 44 may be made of a stainless steel material such as ELGILOY™; or may be made of polymeric material such as PEEK, nylon, polyimide, polyamide, polyethylene or combinations thereof. In one preferred embodiment, the desired properties of structural integrity and torque transmission are achieved by forming the tubular body 44 out of an alloy of titanium and nickel, commonly referred to as nitinol. In a more preferred embodiment, the nitinol alloy used to form the tubular body 44 is comprised of about 50.8% nickel and the balance titanium, which is sold under the trade mark TINEL™ by Memry Corporation. It has been found that a catheter tubular body having this composition of nickel and titanium exhibits an improved combination of flexibility and kink-resistance in comparison to other materials.

Other details regarding construction of balloon guidewire catheters may be found in Assignee's U.S. Pat. No. 6,068,623 and copending applications entitled SHAFT FOR MEDICAL CATHETERS, Ser. No. 09/026,105, filed Feb. 19, 1998; FLEXIBLE CATHETER, Ser. No. 09/253,591, filed Feb. 22, 1999; and FLEXIBLE CATHETER WITH BALLOON SEAL BANDS, Ser. No. 09/653,217, filed Aug. 31, 2000; all of which are hereby incorporated by reference herein in their entirety.

As illustrated in FIGS. 2A and 2B, an expandable member such as the inflatable balloon 12 is mounted on the distal end 48 of tubular body 44. In one preferred embodiment, the balloon 12 is a compliant balloon formed of a material comprising a block polymer of styrene-ethylene-butylene-styrene (SEBS), as disclosed in Assignee's copending application entitled BALLOON CATHETER AND METHOD OF MANUFACTURE, application Ser. No. 09/026,225, filed on Feb. 19, 1998, and in U.S. Pat. No. 5,868,705, the entirety of both of which are hereby incorporated by reference herein. The balloon 12 may be secured to the tubular body 44 by any means known to those skilled in the art, such as adhesives or heat bonding. For example, for attachment of a SEBS balloon to a nitinol tube, a primer such as 7701 LOCTITE™ by Loctite Corporation is preferably used along with cyanoacrylate adhesive such as LOCTITE-4011.

The balloon 12 described in the preferred embodiments preferably has a length of about 5 mm to about 9 mm and more preferably about 6 mm to about 8 mm. Other expandable members are suitable for the catheter 14, such as those disclosed in Assignee's copending applications entitled OCCLUSION OF A VESSEL, Ser. No. 09/026,106, filed Feb. 19, 1998; OCCLUSION OF A VESSEL, Ser. No. 09/374,741, filed Aug. 13, 1999; OCCLUSION OF A VESSEL AND ADAPTER THEREFOR, Ser. No. 09/509,911, filed Feb. 17, 2000; MEMBRANES FOR OCCLUSION DEVICE AND METHODS AND APPARATUS FOR REDUCING CLOGGING, Ser. No. 09/505,554, filed Feb. 17, 2000; and STRUT DESIGN FOR AN OCCLUSION DEVICE, Ser. No. 09/505,546, filed Feb. 17, 2000; the entirety of each of which is hereby incorporated by reference herein.

With reference to FIG. 2B, a core wire 54 is provided inside the lumen 50 and is crimped to the tubular body 44. Coils 56 extend from the distal end 48 of the tubular body 44, surround the core wire 54, and terminate in a distal ball 58. In one embodiment, the core wire 54 may have one or more tapers, and can extend proximally into the tubular body 44. Other details regarding the core wire are discussed in Assignee's copending application, entitled CATHETER CORE WIRE, Ser. No. 09/253,971, filed Feb. 22, 1999, the entirety of which is hereby incorporated by reference.

In one embodiment, shown in FIG. 2B, the tubular body 44 preferably has cuts 60 to create a coiled configuration. A sleeve 62 is preferably provided over the tubular body 44. Adhesive stops 64 and 66 are provided about 1 mm to about 2 mm from the ends of the balloon 12, to control the wicking length of the adhesive 68 into the balloon working area. Balloon inflation is provided through the cuts 60 in the tubular body 44. A marker 70 is mounted to the tubular body 44 proximal of the balloon 12. Adhesive tapers 72 and 74 are provided adjacent the balloon 12 to provide a transition region between the tubular body 44 and the balloon 12 at the balloon's proximal end and between the balloon 12 and the core wire 54 at the balloon's distal end. Seal bands 76 and 78 are respectively applied to the proximal and distal ends of the balloon 12 to improve bond integrity. Other details regarding this embodiment of the balloon catheter 14 may be found in the above-referenced copending applications entitled FLEXIBLE CATHETER and FLEXIBLE CATHETER WITH BALLOON SEAL BANDS.

3. Inflation Adapter and Low Profile Catheter Valve

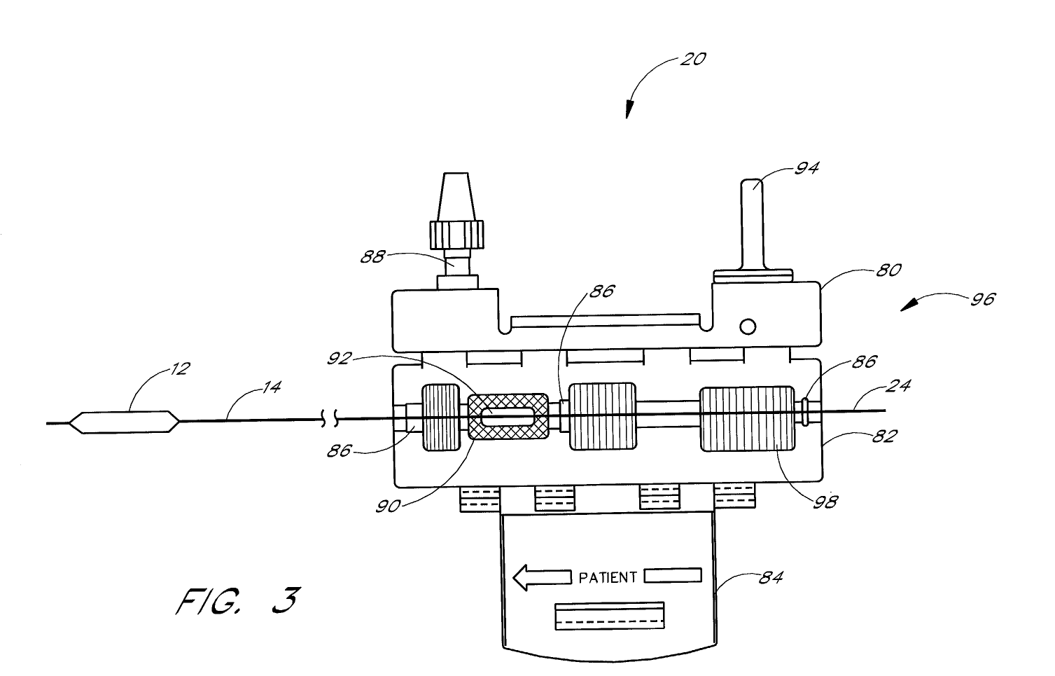

Referring next to FIG. 3, the inflation adapter 20 comprises a housing 96 having two halves 80, 82 preferably formed of metal, medical grade polycarbonate, or the like. The halves 80, 82 are attached by hinges to be separated or joined in a clam shell manner. A locking clip 84 secures the halves 80, 82 while the adapter 20 is in use. Clips 86 within the housing 96 accept and securely hold the catheter 14 in a correct position. The male luer member 88, or another suitable connector, extends from a top of the housing 96 to provide an inflation passageway. Seals 90 are provided within the housing and around an internal segment 92 of the inflation pathway to conduct the pressurized fluid provided by the syringe assembly 22. An actuator 94, shown most clearly in FIG. 1, at the top of the adapter housing 96 controls a cam which operates sliding panels 98 (FIG. 3) contained within the housing 96.

As shown in FIG. 2A, a low profile catheter valve 24 is attached to the open proximal end 46 of the catheter 14. Inflation fluid is injected through the adapter 20 and valve 24 into the lumen 50 (FIG. 2B) of the hollow catheter 14, and into the balloon 12. The inflation adapter 20 is used to open and close the valve 24 to regulate the inflation of the balloon 12 mounted on the distal end 48 of the catheter 14.

It will be emphasized that other types of adapters and/or valves can be employed with the inflation syringe and/or syringe assembly described herein, in order to achieve rapid and accurate inflation/deflation of medical balloons or other non-balloon medical devices. Therefore, although the preferred embodiments described herein are illustrated in connection with a low volume occlusion balloon 12, other types of balloons and non-balloon devices can benefit from the advantages described herein.

As shown in FIGS. 4A and 4B, the low profile catheter valve 24 comprises a movable sealer portion 100 attached at a distal end of a wire segment 102 and positioned within the inflation lumen 50 of the guidewire catheter 14. The wire 102 may be secured to a spring just within the proximal opening 46 of the catheter 14. It will be noted that various spring or biasing arrangements may be utilized, including a zig-zag wire 104 which is formed on or replaces the wire segment 102 and which provides biasing force to the sealer portion 100 due to frictional engagement with the walls of the lumen 50. The sealer portion 100 forms a fluid tight seal with the lumen 50 by firmly contacting the entire circumference of a section of the lumen 50. The sealer portion 100 may be positioned proximally of the side-access inflation port 52 on the catheter 14 as shown in FIG. 4B, to establish an unrestricted fluid pathway between the inflation port 52 and the inflatable balloon 12. As desired, a physician may move the sealer portion 100 to a position at or distal of the inflation port 52, as shown in phantom in FIG. 4B, thereby preventing any fluid from being introduced into or withdrawn from the lumen 50 via the inflation port 52. The valve 24 is considered “low profile” because it is no larger in cross-sectional diameter than the catheter 14 itself.

In operation, the catheter 14 preferably is positioned within the housing 96 of the adapter 20 with the valve 24 closed, such that the side inflation port 52 is located in the sealed inflation area 92 of the housing 96. The catheter 14 is then positioned in the second half 82 of the adapter 20. A distal portion of the catheter 14 extends out of the housing 96 and into the patient, and a proximal portion of the catheter 14 including the catheter valve 24 extends out of the other side of the adapter 20. The adapter 20 is closed, the locking clip 84 is secured, and a syringe assembly 22 is attached (FIG. 1). The actuator 94 is moved from a first position to a second position, such that the sliding panels 98 within the housing 96 cause the valve 24 to be in an open position to allow fluid flow through the inflation port 52. The syringe assembly 22 is then used to inflate the balloon 12. Closing the valve 24 is accomplished by moving the actuator 94 from the second position back to the first position, such that the balloon inflation is maintained. Once the valve 24 is closed the adapter 20 may be removed and treatment and other catheters may be delivered over the guidewire 14.

Other inflation adapter/inflation syringe assemblies may also be used. Also, the adapter 20 can have additional features, such as a safety lock provided on the actuator knob 94 to prevent accidental opening when the adapter 20 is being used and the catheter valve 24 is open. In addition, the adapter 20 can be provided with an overdrive system to overdrive a sealing member into a catheter. Details of these features and other inflation assemblies may be found in Assignee's U.S. Pat. No. 6,050,972 and copending applications, entitled SYRINGE AND METHOD FOR INFLATING LOW PROFILE CATHETER BALLOONS, Ser. No. 09/025,991, filed Feb. 19, 1998; and LOW VOLUME SYRINGE AND METHOD FOR INFLATING SURGICAL BALLOONS, Ser. No. 09/195,796, filed Nov. 19, 1998; all of which are incorporated by reference herein in their entirety.

B. Aspiration Catheter

The occlusion system described above advantageously enables an exchange of catheters over a guidewire 14 while an occlusive device isolates particles within the blood vessel 16. For example, a therapy catheter can be delivered over the guidewire 14 to perform treatment, and then be exchanged with an aspiration catheter to remove particles from the vessel 16. Further details of this exchange are described in Assignee's copending application entitled EXCHANGE METHOD FOR EMBOLI CONTAINMENT, Ser. No. 09/049,712, filed Mar. 27, 1998, the entirety of which is hereby incorporated by reference.

One preferred embodiment of an aspiration catheter 200 is shown in FIG. 5. The catheter 200 includes an adapter 202 and an aspiration port 204 at its proximal end to which a source of negative pressure is attached. The aspiration catheter 200 further comprises an elongate tubular body 206 which extends distally from the adapter 202 and through a pair of support sheaths 210, 212. Beyond the support sheath 212 the elongate tubular body 206 extends to a transition point 214 where the outer diameter of the tubular body 206 tapers down in size. This tapered or necked-down portion of the tubular body 206 is preferably inserted into a proximal end 218 of a dual lumen tubing 216. The tubular body 206 is preferably inserted into one of the lumens of the dual lumen tubing 216 such that its distal end 220 is a sufficient distance distal from the proximal end 218 of the dual lumen tubing 216 to provide a secure connection therebetween.

The dual lumen tubing 216 preferably defines two lumens, one for aspiration and the other for a guidewire to pass therethrough. More particularly, the lumen that the elongate body 206 is inserted into acts as the aspiration lumen, being in fluid communication with the lumen of the elongate tubular body 206. The aspiration lumen preferably ends in a distal aspiration mouth 222, which preferably defines an oblique opening. Aspiration therefore occurs through both the lumen of the elongate tubular body 206 and the aspiration lumen of the dual lumen tubing 216.

The guidewire lumen is provided adjacent the aspiration lumen in the dual lumen tubing 216 and has a proximal end 224 preferably distal to the proximal end 218 of the aspiration lumen of the dual lumen tubing 216, and a distal end 226 preferably distal to the aspiration mouth 222. A marker 228 is placed within the guidewire lumen at the distal end of the aspiration mouth. Additional markers 230, 232 may also be placed over the elongate body 206 and/or support sheaths 210, 212. Further details regarding these and other aspiration catheters are provided below and in Assignee's copending application entitled ASPIRATION CATHETER, Ser. No. 09/454,522, filed Dec. 7, 1999, and U.S. Pat. No. 6,152,909, the entirety of both of which are hereby incorporated by reference.

### II. Aspiration Catheters

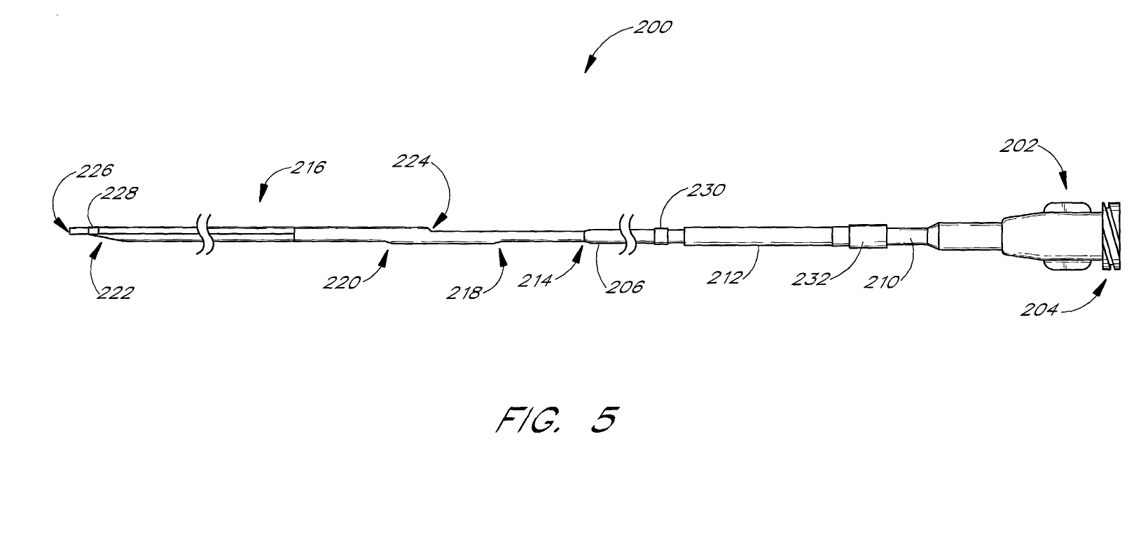

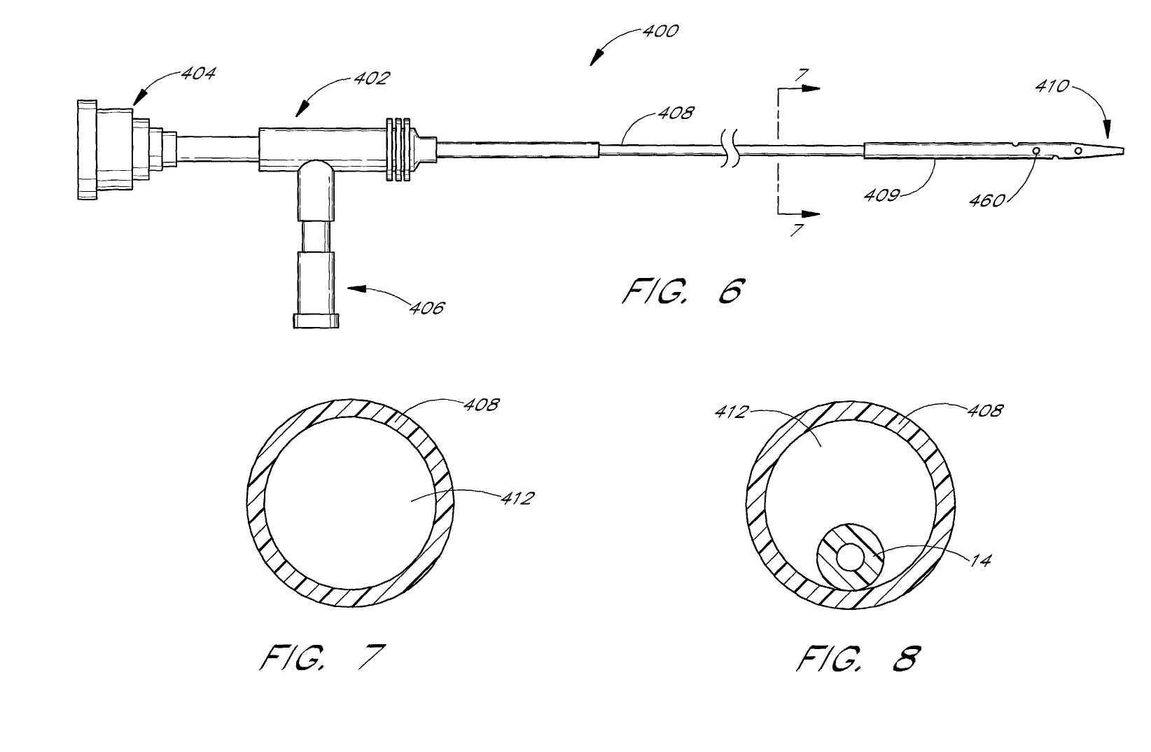

Various aspiration catheters particularly suited for use in the treatment and removal of occlusions in blood vessels as described above are illustrated in FIGS. 6-26. One such aspiration catheter 400, illustrated in FIG. 6, includes an adapter 402, preferably a female luer adapter, and a seal 404 at its proximal end. The catheter 400 further includes an aspiration port 406 to which a source of negative pressure is attached. The aspiration catheter 400 further comprises a long tubular body 408 having a distal end 409 which has a tip 410. The distal end 409 can include a radiopaque marker to aid in locating the tip 410 during insertion into a patient, and is preferably soft to prevent damage to the patient's vasculature. The aspiration catheter 400 is preferably about 145 cm in length, although this length can be varied as desired.

The aspiration catheter 400 illustrated in FIG. 6 is an over-the-wire catheter. As seen in FIG. 7, the tubular body 408 of the catheter 400 is hollow, with an internal diameter ranging from about 0.030 inches to about 0.070 inches. Preferably, the inner diameter is about 0.045 inches. During insertion of the aspiration catheter 400, the proximal end of a guidewire 14 is inserted into the tip 410 of the aspiration catheter 400, and the aspiration catheter 400 is slidably advanced over the guidewire 14, which is positioned inside a hollow lumen 412 of the aspiration catheter 400. The position of the guidewire 14 relative to the tubular body 408 of the aspiration catheter 400 is illustrated in FIG. 8, but of course can vary. For this type of aspiration catheter 400, a very long guidewire 14, generally around 300 cm in length, is used to facilitate passage of the aspiration catheter 400 over the guidewire 14.

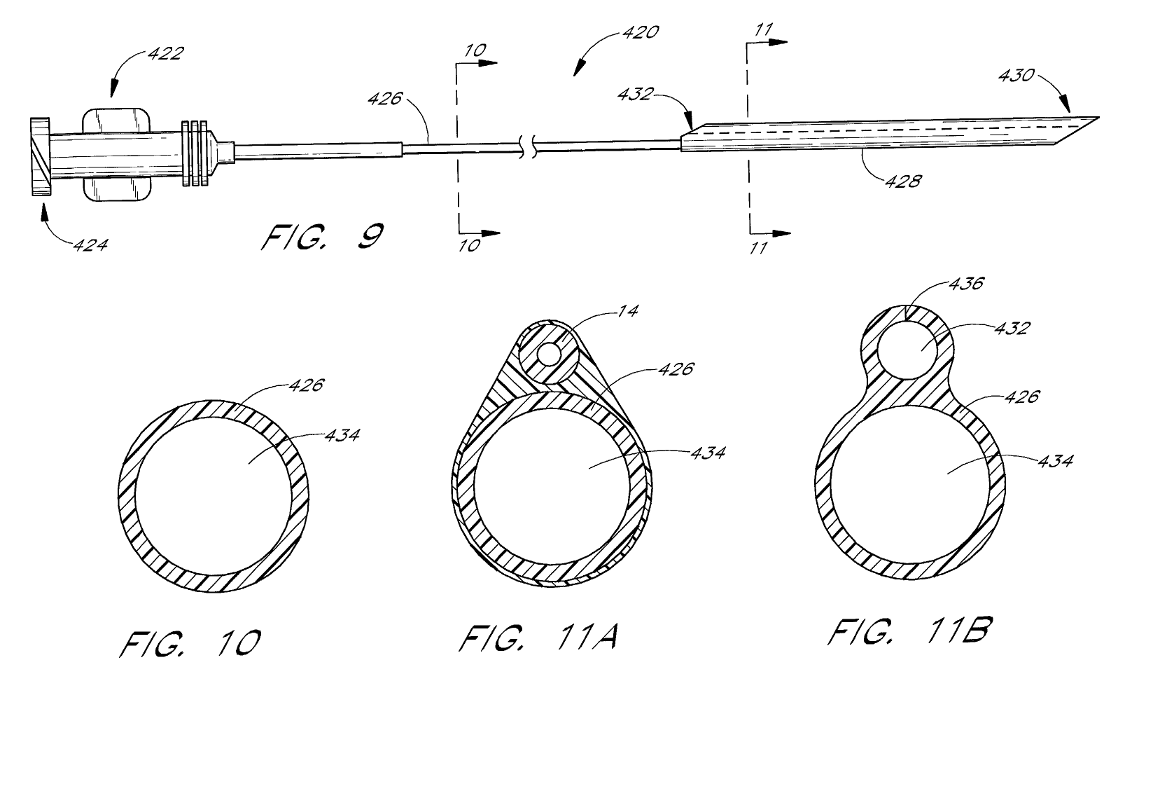

Alternatively, an aspiration catheter 420 can be of a single-operator design, as illustrated in FIGS. 9-11B. The catheter 420 has an adapter 422 and an aspiration port 424 at its proximal end. Like the over-the-wire aspiration catheter 400, the single-operator aspiration catheter 420 comprises a long tubular body 426 having a distal end 428 which has a tip 430. The distal end 428 can include a radiopaque marker to aid in locating the tip 430 during insertion into a patient, and is preferably soft to prevent damage to the patient's vasculature. At the distal end of the tubular body 426 is a guidewire lumen 432. This guidewire lumen 432 provides a separate lumen, apart from a main aspiration lumen 434 of the catheter 420, for insertion of the guidewire 14. The inner diameter of the guidewire lumen 432 ranges from about 0.016 inches to about 0.020 inches for use with a 0.014-inch guidewire system. In a preferred embodiment, the inner diameter of the lumen 432 is about 0.019 inches. As illustrated in FIG. 11A, during delivery of the aspiration catheter 420, the proximal end of the guidewire 14 is inserted into the distal end of the guidewire lumen 432, and the guidewire lumen 432 is slidably advanced over the guidewire 14. Unlike the over-the-wire catheter 400 described above, only a short segment of the single-operator aspiration catheter 420 rides over the guidewire 14, and the guidewire 14 remains in the guidewire lumen 432 and does not enter the main aspiration lumen 434 of the aspiration catheter 420. With the single-operator catheter 420, the long guidewire 14 used with the over-the-wire catheter 400, and the extra operator needed to handle it, are not required.

Although the guidewire lumen 432 is shown in FIG. 9 as being located only on the distal end 428 of the shaft of the aspiration catheter 420, the lumen 432 can also be made to extend the entire length of the shaft 426 if desired. In other embodiments, the guidewire lumen 432 can be less than 10 cm in length; but in still other embodiments, the lumen 432 can extend 30 cm or longer in a proximal direction. In each of these embodiments, however, the aspiration lumen 434 is advantageously left completely unobstructed to provide more efficient aspiration. The guidewire lumen 432 can also include a slit 436 along the entire length in the outside wall of the lumen 432 as shown in FIG. 11B. The slit 436 facilitates faster and easier insertion and removal of the guidewire 14 through the side wall of the lumen 432. By inserting and removing the guidewire 14 through the side wall of the aspiration catheter 420, the need to remove adapters and attachments from the proximal end of the guidewire lumen 432 prior to slidably advancing or removing the aspiration catheter 420 over the guidewire 14 is eliminated.

As will be appreciated by those skilled in the art, in both the over-the-wire and single-operator type aspiration catheters 400, 420, the elongate tubular body of the catheter must have sufficient structural integrity, or “stiffness,” to permit the catheter to be pushed through the vasculature to distal arterial locations without buckling or undesirable bending of the tubular body. It is also desirable, however, for the tubular body to be fairly flexible near its distal end, so that the tubular body may be navigated through tortuous blood vessel networks. Thus, in one preferred embodiment, the tubular body 426 of the aspiration catheter 420 is formed from a polymer such as polyethylene or PEBAX (Atochem, France) made to have variable stiffness along its length, with the proximal portion of the tubular body 426 being less flexible than the distal portion of the tubular body 426. A tubular body of this construction advantageously enables a physician to more easily insert the tubular body into vascular networks that are otherwise difficult to access using conventional catheters of uniform stiffness. This is because the stiffer proximal portion provides the requisite structural integrity needed to advance the catheter without buckling, while the more flexible distal region is more easily advanced into and through tortuous blood vessel passageways.

In one preferred embodiment, variable stiffness along the length of the tubular body of the catheter is achieved by forming a polymeric tubular body which incorporates a reinforcement along its length. For example, the tubular body may be provided with a reinforcing braid or coil incorporated into its wall structure. The reinforcement can be formed of metal or of various polymers. To achieve variable stiffness, the distal region of the catheter is provided with a braid or coil having a higher braid or coil density than that present in the braid or coil of the proximal region. The lower braid density in the proximal region makes it less flexible, or “stiffer,” than the distal region of the catheter.

The precise density of the braiding or coiling provided to the proximal, distal and transition regions can be varied considerably at the time of manufacture, such that catheters having a variety of different flexibility profiles may be created. Moreover, the braid or coil density may be varied within the catheter regions as well, by providing a braid or coil which has a braid or coil density gradient along its length. For example, the proximal-most part of the proximal region may be provided with a metallic braid having a braid density of about 50-90 picks per inch, with the braid density increasing at a rate of about 2-5 picks per inch as the braid extends in the distal direction. This reinforced construction of the catheter provides adequate proximal stiffness for axial push, while preventing collapse of the distal tip during aspiration.

A variety of different materials, known to be ductile and shapeable into fine wires, may be used to form the reinforcement. For example, various polymers, stainless steel, silver or gold plated stainless steel, platinum, nitinol, or a combination thereof are suitable. In one preferred embodiment, the braid is formed of stainless steel, and has a braid density which varies from 50-70 picks per inch at the most proximal part of the proximal region of the catheter, to 80-100 picks per inch at the most distal part of the distal region of the catheter.

Reinforcing braids or coils may be introduced into the structure of the catheter body through conventional catheter forming techniques. For example, the tubular body may be formed by inserting a 72D PEBAX tube into a variable braid density stainless steel sleeve, and then inserting the sleeved tube into a 72D PEBAX outer tube of the same length, so that the braided sleeve is sandwiched between the two tubes. A shaping mandrel may be inserted within the inner PEBAX tube, and shaping container over the outer PEBAX tube, and the entire apparatus may then be placed in a hot box kept at a temperature slightly greater than the melting temperature of the PEBAX tubes. The PEBAX tubes will melt and fuse together, and once cooled, will form a tubular body incorporating the braid. This same technique can be used to form a tubular body incorporating a coil.

In another embodiment, variable stiffness of the tubular body may be achieved by forming the proximal and distal regions of the tubular body out of polymeric materials having differing degrees of stiffness. For example, one half of an inner tube of 72D PEBAX may be inserted into an outer tube of 40D PEBAX, and the other half of the inner tube may be inserted into a 72D PEBAX outer tube. The combination may then be heat fused, as described above. The 40D/72D PEBAX combination forms a more flexible tubular body than the region of the 72D/72D PEBAX combination. More or less flexible materials may be used as desired to alter the flexibility of the resulting tubular body. Furthermore, the flexibility of the various regions of a tubular body formed in this manner may be varied further by incorporating a braid or coil having either a uniform braid density or coil pitch, or a varying density or coil, into the tubular body, as described above.

Moreover, any of a variety of different polymeric materials known by those of skill in the art to be suitable for catheter body manufacture may be used to form the catheter body. For example, the body may be formed out of polymers such as polyethylene, PEBAX, polyimide, polyether etherketone, and the like. Different materials might also be combined to select for desirable flexibility properties.

Also, although the catheter body has been described in the context of having two regions of differing flexibility, it will be readily appreciated by those of skill in the art that three or more regions of differing flexibility may easily be provided, by adapting the teachings contained herein.

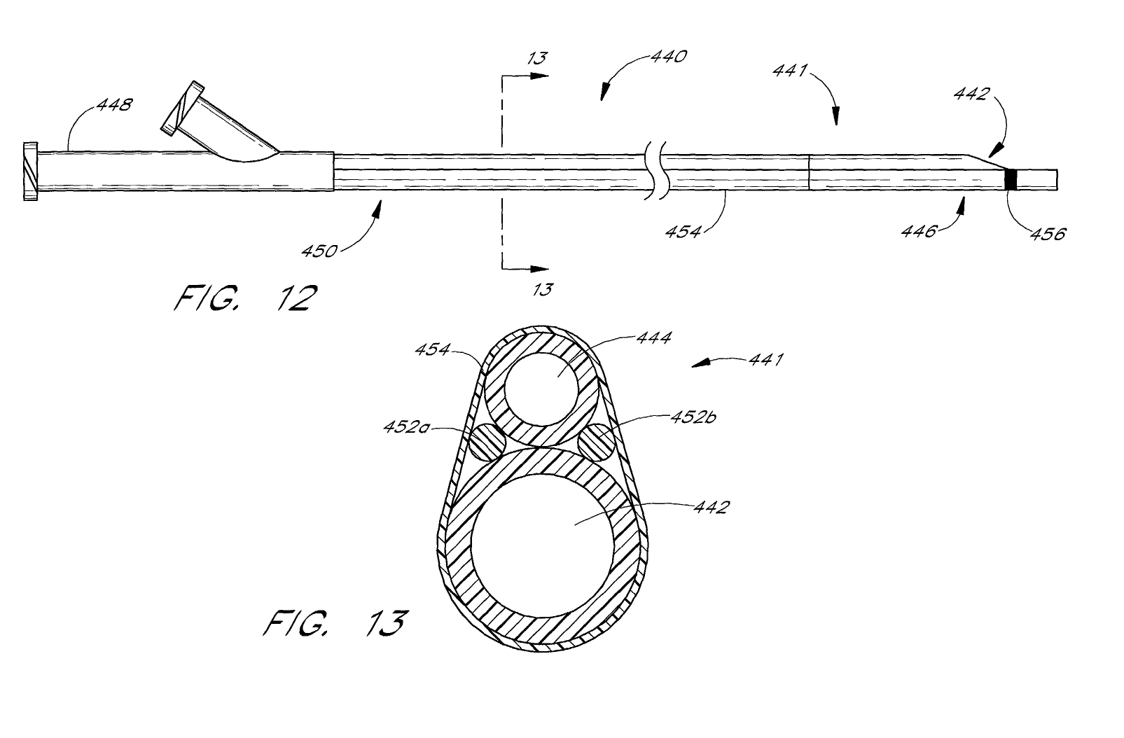

A further embodiment of an aspiration catheter includes at least one support mandrel incorporated into the catheter body to further strengthen the catheter. One such aspiration catheter 440, having two support mandrels, is illustrated in FIG. 12. This over-the-wire aspiration catheter 440 is approximately 135-140 cm in length, and includes a tubular body 441 comprising an aspiration lumen 442 and a separate guidewire lumen 444. Both the lumens 442, 444 extend from a proximal end 450 of the catheter 440 to a distal end 446. As explained above with reference to the catheters 400, 420, the catheter 440 preferably includes an adapter 448 at the proximal end 450. The adapter 448 connects to a source of negative pressure to provide aspiration through the aspiration lumen 442. During insertion into a patient's vasculature, the catheter 440 is slidably advanced over a guidewire 14 positioned within the guidewire lumen 444, as described above.

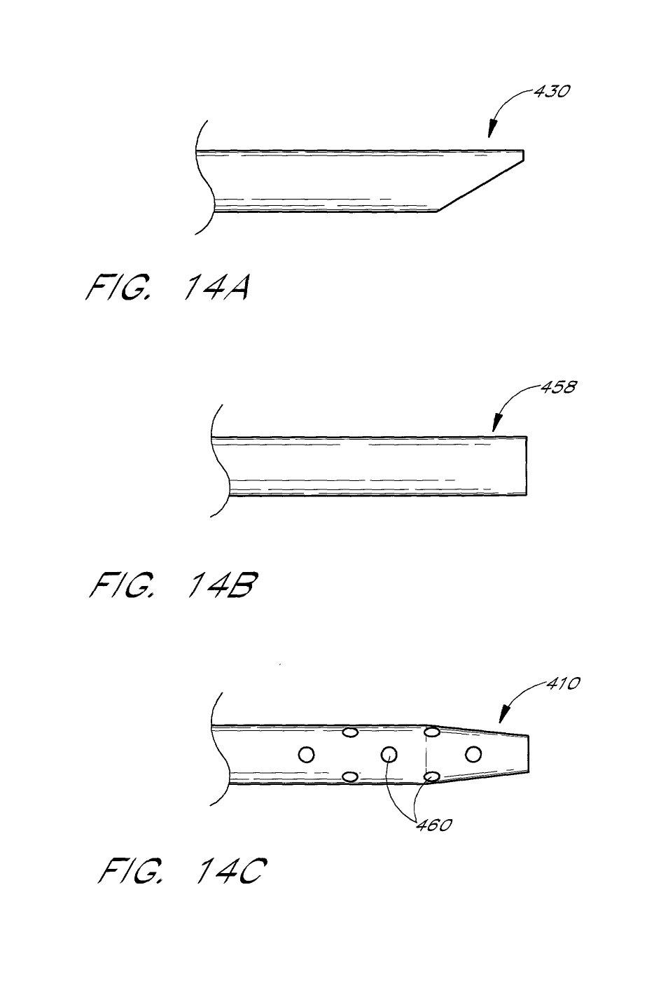

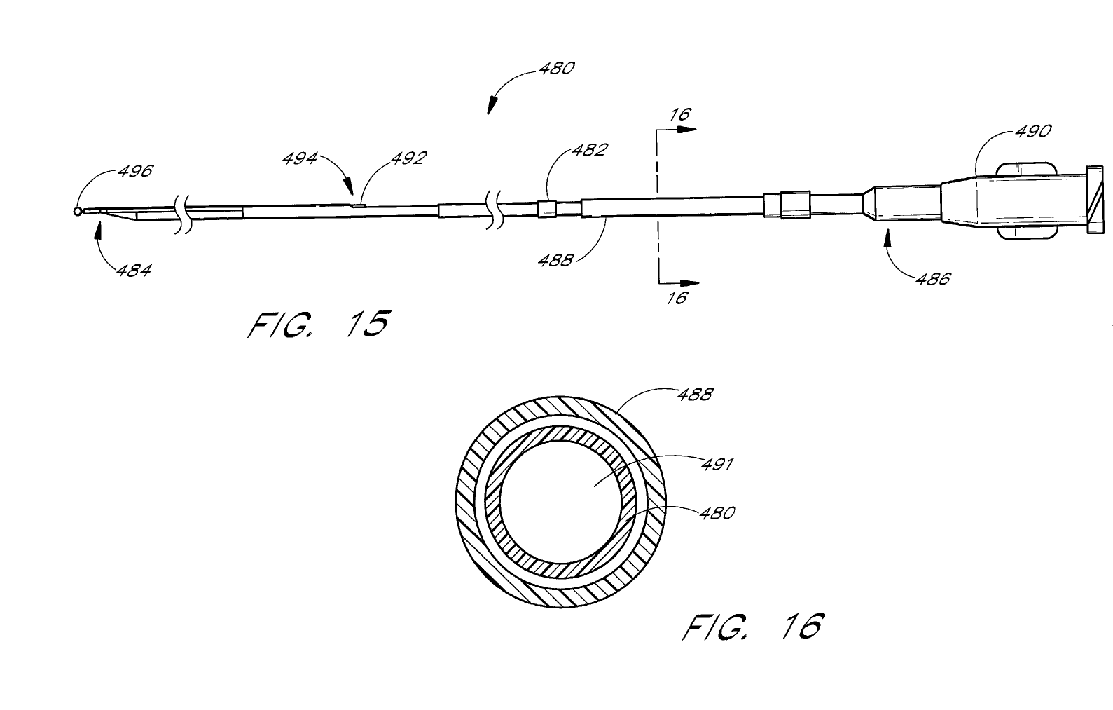

As illustrated in FIG. 13, the aspiration and guidewire lumens 442, 444 are adjacent to one another, with two support mandrels 452a, 452b positioned alongside the lumens 442, 444 to provide added stiffness to the length of the tubular body 441. The mandrels 452a, 452b are optional, and are preferably formed of stainless steel, but could be made of any material that would provide additional strength to the tubular body 441. The outer diameter of each of the mandrels 452a, 452b is preferably no more than about 0.010 inches, to maintain the low profile of the tubular body 441. The mandrels 452a, 452b extend from the proximal end 450 of the tubular body 441, ending approximately 35 cm from the distal end 446 of the catheter.

As is illustrated in FIG. 13, a shrink tube 454 surrounds the dual lumen tubing 442, 444 and the mandrels 452a, 452b. The shrink tube 454 is formed of polyethylene terephthalate (PET) or other suitable material. During manufacture of the catheter 440, the shrink tube 454 tightens around the dual lumen tubing 442, 444 and the mandrels 452a, 452b, maintaining the position of the mandrels 452a, 452b adjacent of the lumens 442, 444 along the length of the tubular body 441. The shrink tube 454 extends approximately 10 cm beyond the ends of the mandrels 452a, 452b at the proximal end 450 of the catheter 440, to secure the shrink tube 454 around the tubular body 441 and prevent the mandrels 452a, 452b from moving. The shrink tube 454 therefore extends from the proximal end 450 of the catheter 440 to a position approximately 25 cm from the distal end 446 of the aspiration catheter 440.

The distal end 446 of the aspiration catheter 440 preferably is formed from 25D to 40D PEBAX with a radiopaque filler such as BaS0₄. Alternatively, the distal end 446 of the catheter 440 can also be provided with a soft distal tip which is not pre-formed with the tubular body 441, but instead is attached to the tubular body 441 as a post manufacturing step. The distal end 446 preferably is soft enough and flexible enough so as to minimize trauma to body vessels as the catheter 440 is advanced and to facilitate navigation of the catheter 440 in tortuous vessels, but the distal end 446 must also be strong enough to avoid collapse during aspiration. In one preferred embodiment, the distal end 446 is formed as a 0.5 cm sleeve of 25-35D PEBAX and is bonded to the tubular body 441 by use of an adhesive. Alternately, the distal end 446 may be attached to the tubular body 441 by heat bonding, as is known to those of skill in the art.

The entire distal end 446 of the aspiration catheter 440 can also be attached as a separate post manufacturing step. A tubing made of polyethylene (PE), PEBAX, or polyimide can be fused to the distal end of the main tubular body section of the catheter. This tubing can be from about 5 cm to about 60 cm in length, but is preferably around 30 cm in length. The distal end 446 of the aspiration catheter 440 can also be provided with a radiopaque material. Advantageously, radiopaque material serves as a marker 456 to help the physician position the catheter 440 inside the patient's body. Various well-known radiopaque materials may be used in the distal end 446 to form the marker 456, such as platinum or gold. Alternatively, BaS0₄ can be incorporated into the polymer resin itself.

FIGS. 14A-14C illustrate various embodiments of distal tips that may be incorporated into the aspiration catheters described herein. FIG. 14A shows a preferred distal tip 430, wherein the tip 430 has been angled and is oblique to maximize the drug delivery and aspiration areas, and to provide effective retrieval of particles. The oblique distal tip 430 also minimizes the risk of the aspiration catheter sucking on the vessel wall 16 which can cause trauma to or disruption of the vessel intima, and/or sealing of the hole at the tip of the aspiration catheter to the balloon 12. Furthermore, this distal tip 430 maximizes the area of aspiration. The angle can be from about 5 degrees to about 90 degrees; an angle of about 25 degrees is preferred. The distal tip 430 is also incorporated into the aspiration catheter 420, as shown in FIG. 9. As shown in FIG. 14B, the distal end of the aspiration catheter can also comprise a blunt tip 458, or a tapered tip 410 as shown in FIG. 14C. Side ports 460 can be drilled along the distal tip of the catheter to enhance the aspiration rate, as illustrated in FIGS. 6 and 14C.

The proximal end of the aspiration catheter may also include a marker 482 which indicates to the physician the approximate catheter length which can safely and rapidly be inserted into the patient. This femoral marker 482 is illustrated as part of an aspiration catheter 480 in FIG. 15. The marker 482 is placed on the aspiration catheter 480 approximately 95 cm from a distal tip 484 of the catheter 480. This length is approximately equal to the distance from the incision site in the femoral artery to the ostium of the coronary artery in the average human being. Thus, during insertion of the catheter 480, the physician rapidly advances the catheter 480 into the patient's vasculature, until the femoral marker 482 near the proximal end of the catheter 480 is just outside the patient's body. At this point, the marker 482 is an indication to the physician to slow the insertion of the catheter 480, and to turn on the fluoroscopy to carefully deliver the distal tip 484 of the catheter 480 to the desired position. This therefore reduces the patient's exposure to x-rays during the procedure. The femoral marker 482 may be made of any biocompatible material, including plastics and metals, however, any visible marker 482 on the outer surface of the catheter 480 may be used.

As illustrated in FIG. 15, the aspiration catheter 480 may include one or more adapters 490 and valves. For example, commonly used adapters and valves include a Touhy-Borst or hemostasis valve (not shown), which is positioned at the proximal end 486 of the aspiration catheter 480. The hemostasis valve surrounds the outer surface of the aspiration catheter 480, and tightens down around the aspiration catheter 480 to prevent the patient's blood from flowing out around the aspiration catheter 480. As the valve is tightened, there is some risk that the aspiration catheter 480 may be crushed. Accordingly, a support sheath 488 is positioned near the proximal end 486 of the catheter 480, just proximal of the adapter 490, to prevent collapse or crushing of the catheter 480. As shown in FIG. 16, the sheath 488 surrounds the outer surface of the aspiration catheter 480, giving that portion of the aspiration catheter 480 greater strength and preventing the valve from crushing an aspiration lumen 491 of the catheter 480.

In addition, the support sheath 488 allows the aspiration catheter 480 to move within the rotating hemostasis valve. As described below, the physician may wish to move the distal tip 484 of the aspiration catheter 480 in a proximal direction during aspiration, to ensure complete aspiration of debris. If the hemostasis valve is tightened directly onto the aspiration catheter 480, the catheter 480 is not free to slide back and forth. If the valve is tightened on the support sheath 488, however, there is a sufficient gap (FIG. 16) between the support sheath 488 and the body of the aspiration catheter 480 to allow for slidable movement of the aspiration catheter 480. The support sheath 488 preferably is formed of polyimide and has a length of about 3-9 cm; more preferably about 6 cm.

A rod 492 is shown in FIG. 15, extending through a guidewire lumen 494 of the catheter 480. The rod 492 is used when packing and shipping the catheter to prevent the guidewire lumen 494 from collapsing. The rod 492 terminates in a distal ball 496 to hold the rod 492 against the distal end of the guidewire lumen 494.

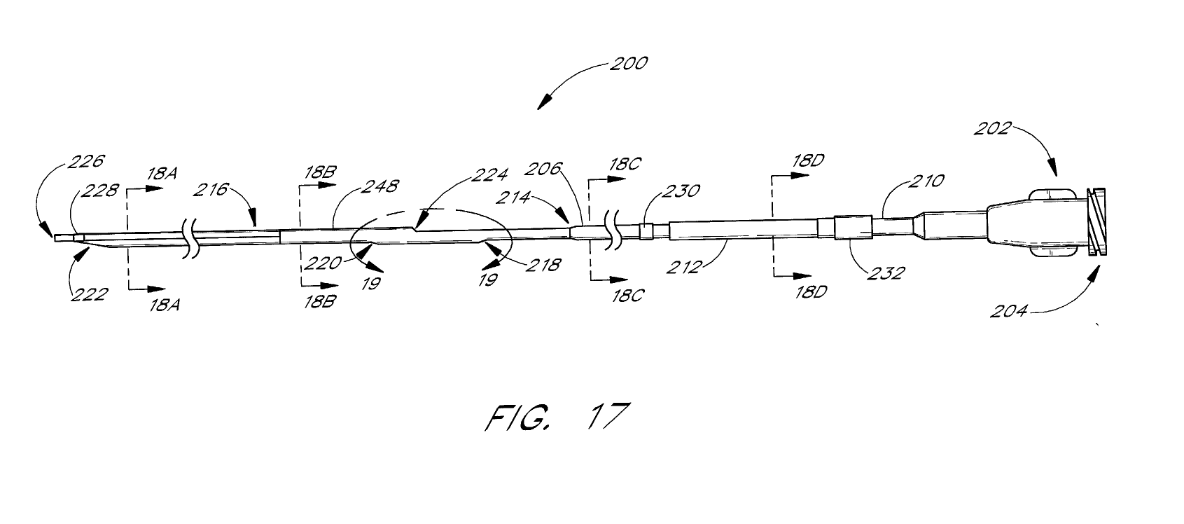

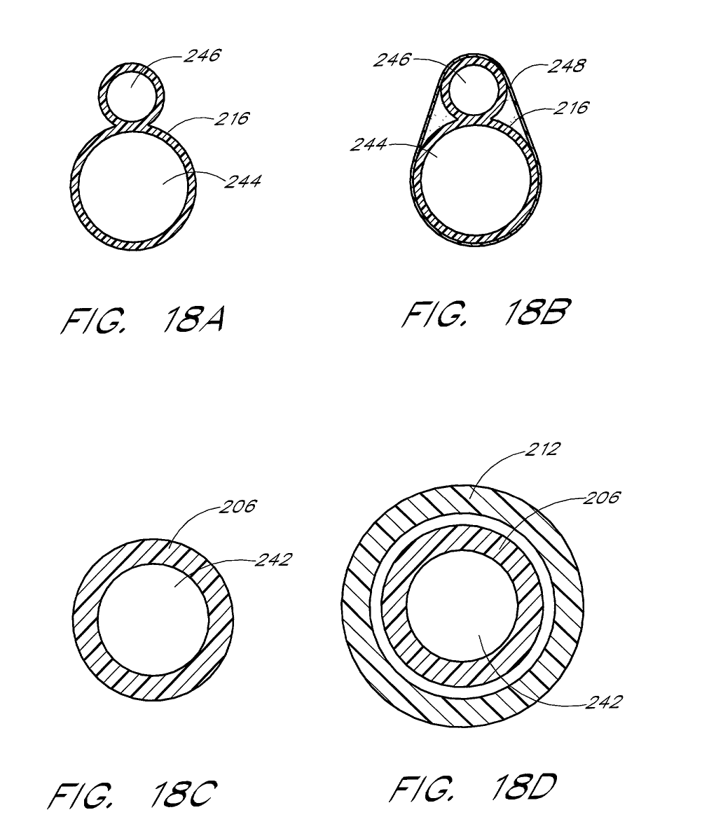

FIG. 17 illustrates another embodiment of an aspiration catheter 200 which is substantially similar to the catheter of FIG. 15, as briefly described with respect to FIG. 5 above. FIGS. 18A-18D show cross-sections at four locations of the aspiration catheter 200. The proximal section of aspiration catheter 200 is a single-lumen design, comprising an elongate tubular body 206 having an aspiration lumen 242 (FIGS. 18C-18D), whereas the distal section of the aspiration catheter 200 is a dual-lumen tubing 216 comprising an aspiration lumen 244 and a guidewire lumen 246.

The guidewire lumen 246 in the catheter 200 preferably extends distally beyond the aspiration lumen 244 by a distance of about 0.5 mm to about 5 mm; more preferably about 1.5 mm. The catheter 200 preferably includes at least three markers 228, 230, and 232 disposed at different locations along the length of the catheter 200. The distal-most marker 228 is preferably provided at the distal end of the dual lumen tubing 216 at the distal tip of the aspiration mouth 222. More preferably, the marker 228 is inserted inside the guidewire lumen 246 at the position of the aspiration mouth 222 of the aspiration lumen 244. One or more markers 230 are placed on the elongate tubular body 206 of the single lumen tubing. For example, a pair of markers 230 may be placed on the elongate tubular body 206, one spaced about 43 cm from distal end 226, the other spaced about 90 cm from distal end 226. A marker 232 may also be placed on a support sheath 210 of the catheter 200.

As with the catheter of FIG. 15 above, as shown in FIG. 17, support sheaths 210 and 212 surround the proximal end of the catheter 200. The support sheath 212 preferably extends from the proximal end of the elongate tubular body 206, and in one embodiment is about 6 cm long. The support sheath 210 preferably extends over sheath 212 from the proximal end of the elongate tubular body 206, and in one embodiment has a length of about 1-2 cm. FIG. 18B illustrates a shrink tubing 248 surrounding the portion of the catheter 200 wherein the elongate tubular body 206 is joined with the dual lumen tubing 216, as described in further detail below.

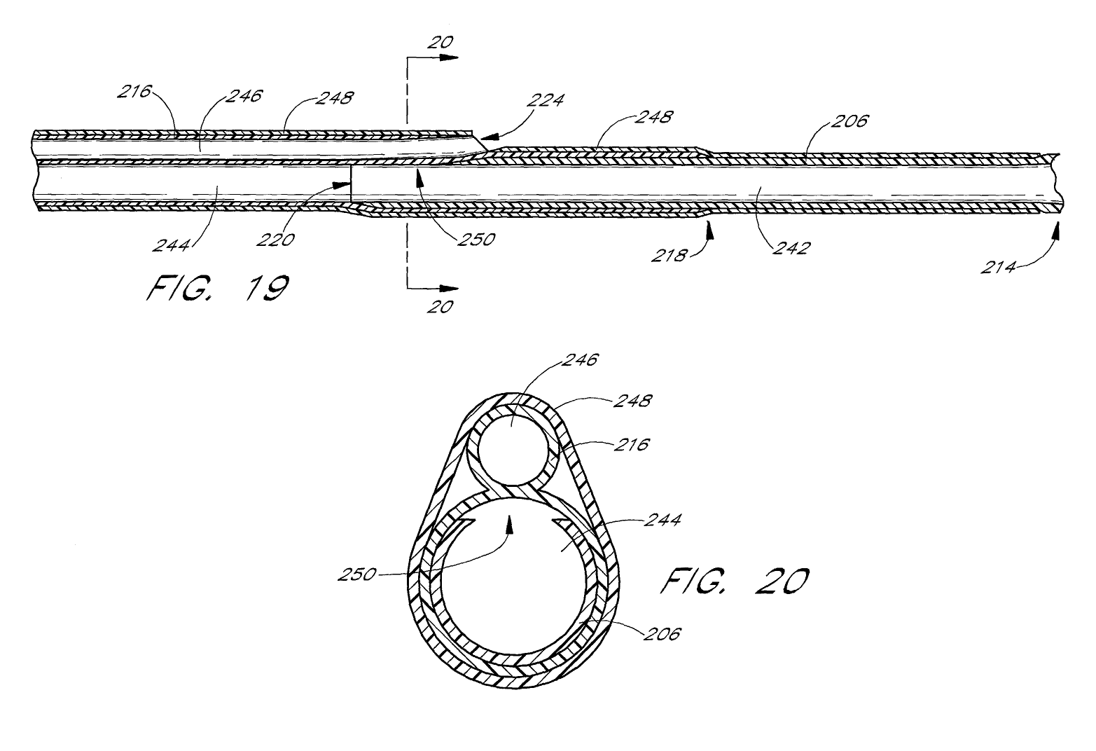

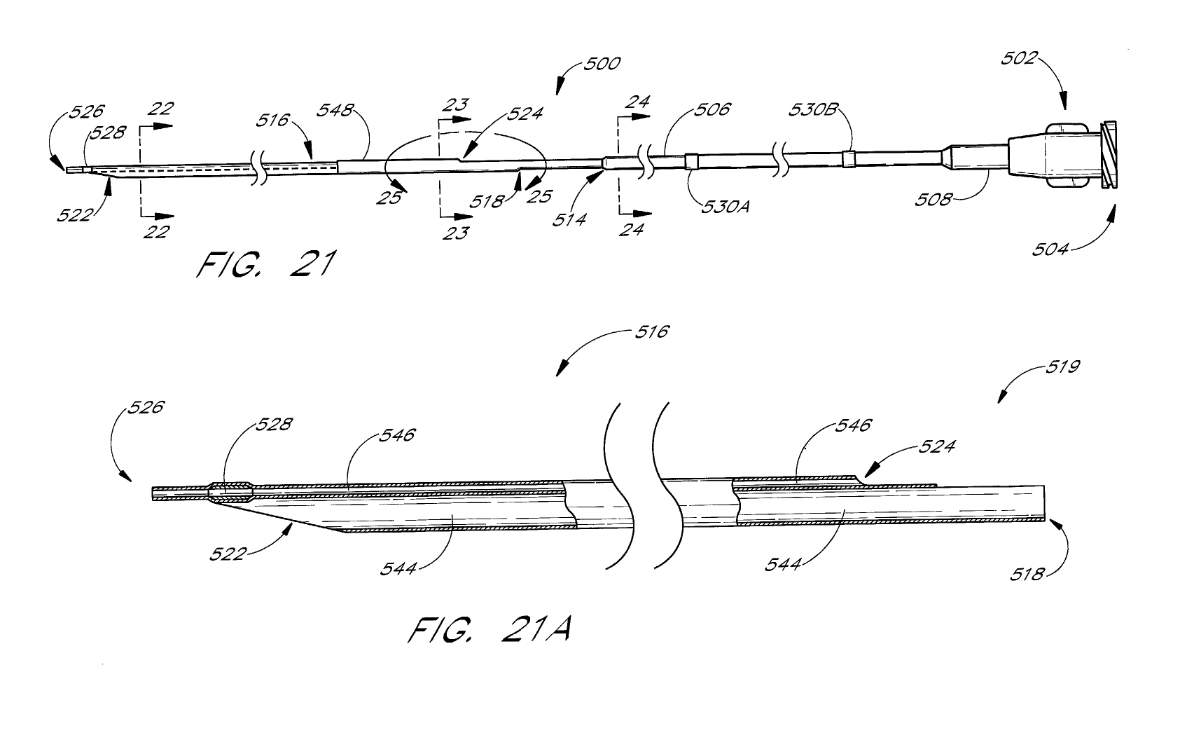

As shown in FIGS. 19 and 20, the aspiration catheter 200 is constructed by joining the single lumen, elongate tubular body 206 with the dual lumen tubing 216. In one embodiment, the dual lumen tubing 216 is integrally formed. However, it will be appreciated that the guidewire lumen 246 can be separately attached to the aspiration lumen 244. To construct the catheter 200, the single lumen tubing 206 is inserted into dual lumen tubing 216 after the single lumen tubing 206 undergoes “necking” and cutting, as described below.

In the necking process, the distal end of the single lumen tubing 206 is stretched while heating to narrow its diameter to allow insertion into the proximal end of the dual lumen tubing 216. This produces a necked portion, as shown in FIG. 19, which extends distally from the transition point 214. In one embodiment, this necked portion is about 10-30 mm in length; and more preferably, the necked portion is about 20 mm in length.

After necking, as shown in FIGS. 19 and 20, the distal end of the “necked” portion of the single lumen tubing 206 is preferably cut or shaved longitudinally to remove a portion of the tubing to allow easier insertion of the single lumen tubing 206 into the dual lumen tubing 216. More preferably, material is removed from the distal end of the single lumen tubing 206 to produce a cut section 250 such as shown in FIG. 20. In a preferred embodiment, when the two pieces of tubing are joined, the cut section 250 faces upward to minimize pressure on the guidewire lumen 246 and to therefore maximize the profile of the guidewire lumen 246. The cut section 250 preferably extends from the distal end of the single lumen tubing 206 by a distance of about 1 mm to about 10 mm, more preferably about 5 mm, corresponding to the length of the single lumen tubing 206 that is inserted into the dual lumen tubing 216.

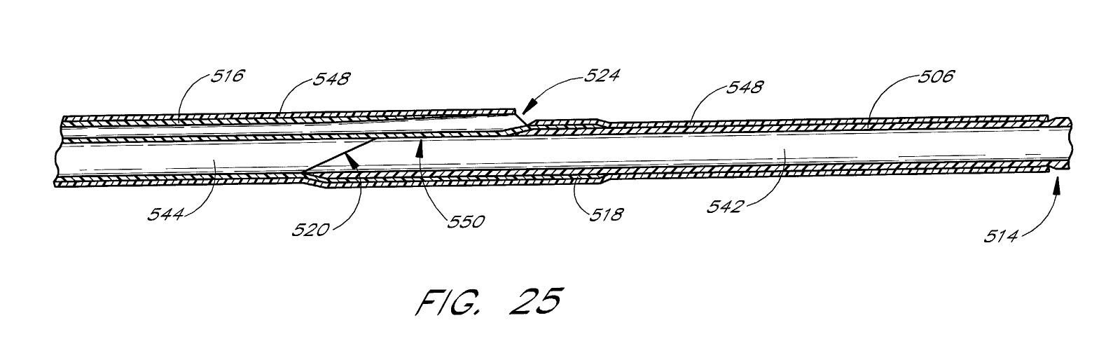

A distal end 220 of the single lumen tubing 206 is illustrated in FIG. 19 as being straight or perpendicular with respect to the longitudinal axis of the single lumen tubing 206. It will be appreciated, however, that the distal end 220 may be given an oblique cut to facilitate insertion of the single lumen tubing 206 into the dual lumen tubing 216. Such an embodiment wherein an oblique distal end 520 is utilized is shown in FIG. 25.

After the single lumen tubing 206 is inserted into the dual lumen tubing 216, an adhesive is applied to hold the two pieces of tubing together. As shown in FIGS. 17 and 18B, a shrink tubing 248 formed of polyethylene terephthalate (PET), or other suitable material, is positioned over the junction of the single lumen tubing 206 and the dual lumen tubing 216. The shrink tubing 248 preferably extends distally from the proximal end 224 of the guidewire lumen 246 by a distance of about 5 mm to 30 mm, more preferably about 15 mm, and proximally from the proximal end of the guidewire lumen 246 by a distance of about 5 mm to about 30 mm, more preferably about 15 mm.

In order to keep the guidewire lumen 246 open during fabrication, a wire mandrel similar to the rod 492 of FIG. 15 can be inserted into the guidewire lumen 246 while the single lumen tubing 206 is inserted into the aspiration lumen 244. Furthermore, such a mandrel preferably extends out of the proximal end 224 of the guidewire lumen 246 while the shrink tubing 248 is being applied, thereby keeping the proximal end 224 of the guidewire lumen 246 exposed.

In one embodiment, the single lumen tubing 206 of the catheter 200 is preferably made of polyimide or PEEK, or a combination thereof. The dual lumen tubing 216 is preferably made of polyethylene. It will be appreciated that other materials may also be used. The junction of the single and double lumen tubing 206, 216 preferably is made as small as possible; and in one embodiment, has an outer diameter of no more than about 0.069 inches. Further details regarding aspiration catheters are described in the above-mentioned U.S. Pat. No. 6,152,909.

FIG. 21 shows another embodiment of an aspiration catheter 500, which is substantially similar to the aspiration catheter 200 of FIG. 17, having a single lumen tubing 506 joined with a dual lumen tubing 516. Similar to the catheter 200, the catheter 500 includes an adapter 502 and an aspiration port 504, attached to a proximal end 508 of the aspiration catheter 500, to which a source of negative pressure can be attached. The length of the dual lumen tubing 516 is such that a junction between the dual lumen tubing 516 and the single tubing 506 remains within the catheter guide tube during aspiration procedures. In one embodiment, the dual lumen tubing 516 has a length between about 35 centimeters and about 36 centimeters. In another embodiment, the dual lumen tubing 516 has a length of about 35 centimeters. When joined, the single and dual lumen tubing 506, 516 preferably are at least 145 cm in length, and the catheter 500 preferably is about 150 cm in length (in absence of the adapter 502). The proximal end 508 preferably has a diameter of about 0.067 inches to about 0.073 inches; more preferably, about 0.070 inches.

As illustrated, the single lumen tubing 506 has two markers 530A and 530B, and no support sheaths. The marker 530A is preferably spaced about 43 cm from a distal end 526 of the catheter 500, and the marker 530B is preferably spaced about 90 cm from the distal end 526. The markers 530A and 530B indicate to the physician the approximate catheter length which can safely and rapidly be inserted into the patient. The marker 530A indicates to the physician when the distal tip of the catheter is about to exit the guide catheter tube. During insertion of the catheter 500, the physician may rapidly advance the catheter 500 into the patient's vasculature, until the marker 530B near the proximal end 508 of the catheter 500 is just outside the patient's body. At this point, the marker 530B is an indication to the physician to slow the insertion of the catheter 500 and to turn on the fluoroscopy to carefully deliver the distal end 526 of the catheter 500 to the desired position. This therefore reduces the patient's exposure to x-rays during the procedure. The markers 530A and 530B may be made of any biocompatible material, including plastics and metals; however, any visible marking on the outer surface of the catheter 500 may be used. It will be appreciated by those of ordinary skill in the art that each of the markers 530A, 530B has a thickness, and thus contributes to the profile of the single lumen tubing 206. Preferably, the markers 530A, 530B are provided with thicknesses such that the profile of the single lumen tubing 206 at the positions of the markers 530A, 530B is no more than about 0.054 inches.

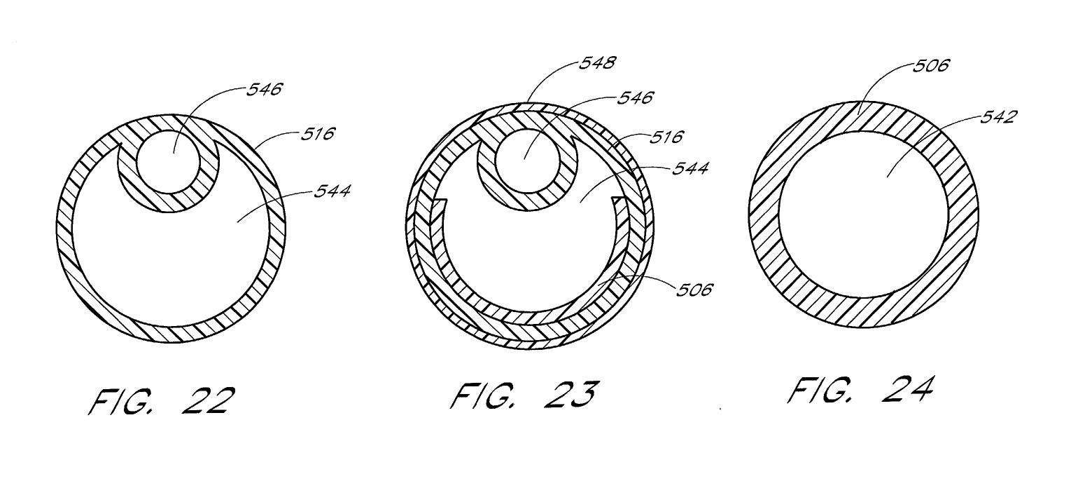

As shown in FIG. 22, a guidewire lumen 546 is located within an aspiration lumen 544 such that the aspiration lumen 544 has a crescent cross-sectional shape and the dual lumen tubing 516 has a round cross-sectional shape. It will be appreciated by those skilled in the art that placing the guidewire lumen 546 within the aspiration lumen 544 advantageously reduces the profile of the dual lumen tubing 516, as compared with the dual lumen tubing 216 of the catheter 200, but also reduces the cross-sectional area of the aspiration lumen 544 to some extent. However, it has been determined that the crescent cross-section of the aspiration catheter 500 provides evacuation flow rates that are similar to the flow rates attainable by use of the catheter 200 (FIG. 17), wherein a circular aspiration lumen 244 is utilized. More specifically, it has been determined that when coupled with a 20-cc aspiration syringe the aspiration catheter 500 preferably provides an evacuation flow rate of about 0.5 cc/second to about 0.9 cc/second, yielding an average flow rate of about 0.7 cc/second. More preferably, the aspiration catheter 500 provides optimal evacuation flow rates that are not less than about 0.68 cc/second, or about 41 cc/minute.

A cross-sectional view at the junction between the single lumen and dual lumen tubing 506, 516 is shown in FIG. 23, and a cross-sectional view of the single lumen tubing 506 having an aspiration lumen 542 is shown in FIG. 24. The single lumen tubing 506 preferably has an outside diameter of about 0.052 inches (4.0 French), and the dual lumen tubing 516 preferably has an outside diameter of about 0.060 inches (4.6 French). In one embodiment, the aspiration lumen 544 has a major diameter (i.e., side to side distance) of about 0.050 inches and a minor diameter (i.e., top to bottom distance) of about 0.029 inches, providing a cross-sectional area of about 0.0018 square inches, and the guidewire lumen 546 has a diameter of about 0.017 inches. In another embodiment, the aspiration lumen 544 has a major diameter of about 0.054 inches and a minor diameter of about 0.032 inches, and the guidewire lumen 546 has a diameter of about 0.018 inches. As described above with reference to the catheter 200, the catheter 500 shown in FIG. 21 is constructed from the single lumen tubing 506 and the dual lumen tubing 516 which are joined as described above, except that the dual lumen tubing 516 has the two concentric lumens 544, 546 rather than adjoining lumens.

As shown most clearly in FIG. 21A, a proximal end 524 of the guidewire lumen 546 is distally spaced from a proximal end 518 of the dual lumen tubing 516, and the guidewire lumen 546 extends distally beyond the aspiration lumen 544, thus forming the distal end 526. The proximal end 524 of the guidewire lumen 546 preferably is spaced from the proximal end 518 by about 1 mm to about 10 mm; more preferably, about 8 mm to about 9 mm. The guidewire lumen 546 preferably extends beyond the aspiration lumen 544 by a distance of about 0.5 mm to about 5 mm; more preferably about 1.5 mm. The distal end 526 of the catheter 500 preferably has a maximum outside diameter of about 0.025 inches.

It will be appreciated that although FIGS. 21 and 21A show the guidewire lumen 546 spanning substantially the entire length of the dual lumen tubing 516, the guidewire lumen 546 can have a length shorter than the length of the dual lumen tubing 516. In one embodiment, the guidewire lumen 546 extends from the distal end 526 to a location on the dual lumen tubing 516 about 6 cm proximal of the distal end 526. It is contemplated, however, that the guidewire lumen 546 can be formed such that it extends from the distal end 526 to any desired location along the dual lumen tubing 516, depending upon the particular procedure for which the catheter 500 is intended to be used. It is further contemplated that, in one embodiment, the guidewire lumen 546 may be shortened by forming the proximal end 524 at a desired location along the dual lumen tubing 516, distal of the proximal end 524 shown in FIGS. 21 and 21A. Alternatively, the guidewire lumen 546 may be shortened by forming a cut section in the side of the dual lumen tubing 516 such that the guidewire lumen 546 is exposed to the exterior of the dual lumen tubing 516.

As illustrated in FIG. 21A, the catheter 500 preferably includes a distalmost radiopaque marker 528 at the distal end 526 of the dual lumen tubing 516, positioned at the distal edge of an aspiration mouth 522 of the aspiration lumen 544. More preferably, the marker 528 is inserted inside the guidewire lumen 546 and positioned at the position of the distal edge of the aspiration mouth 522. The marker 528 facilitates visualization of the location of the opening of the aspiration lumen 544 while advancing/retracting the aspiration catheter 500 within the patient. As will be appreciated by those skilled in the art, knowing the location of the opening of the aspiration lumen 544 within the patient enables the physician to avoid advancing the distal end 526 of the catheter 500 into the balloon 12. This substantially eliminates the risk of damaging the balloon 12 and the distal tip 526, as well as forcible movement of the balloon 12 while it is inflated within the vessel 16.

As with the catheter 200 shown in FIG. 17, the catheter 500 shown in FIG. 21 is constructed from single lumen tubing 506 inserted into dual lumen tubing 516 after “necking” and shaving a region, as discussed below. As illustrated in FIG. 25, the single lumen tubing 506 is inserted into the dual lumen tubing 516, with the single lumen tubing 506 having a necked portion distal of a transition area 514 and a cut portion 550 at the distal end 520 of the necked portion. This necked portion of the single lumen tubing 506 is preferably inserted into the dual lumen tubing 516 through the proximal end 518 of the dual lumen tubing 516. In one embodiment, the necked portion preferably is about 30 mm in length, and has an outside diameter of about 0.040 inches to about 0.042 inches; more preferably, about 0.041 inches.

The distal end 520 of the single lumen tubing 506 may be straight or oblique; and as shown in FIG. 25, the distal end 520 is preferably oblique. The oblique distal end 520 preferably comprises an angle between about 10° and about 45° with respect to the longitudinal axis of the catheter 500; more preferably, the angle is about 30°. The oblique distal end 520 facilitates insertion of the single lumen tubing 506 into the dual lumen tubing 516.

As mentioned above, the distal end 520 of the single lumen tubing 506 is preferably cut or shaved longitudinally to remove a portion of the tubing to facilitate easier insertion of the single lumen tubing 506 into the dual lumen tubing 516. More specifically, material is removed from the distal end 520 of the single lumen tubing 506 to form the cut portion 550 as shown in FIG. 25. Preferably, enough material is removed to provide the distal end 520 with a minor diameter of about 0.035 inches to about 0.037 inches; more preferably, about 0.036 inches. In a preferred embodiment, when the two pieces of tubing 506, 516 are joined, the cut portion 550 is faced toward the guidewire lumen 546 such that the single lumen tubing 506 exerts minimal pressure on the guidewire lumen 546 and thus maximizes the profile thereof. The cut section 550 extends from the distal end 520 of the single lumen tubing 506 by a distance corresponding to the length of the single lumen tubing 506 that is inserted into the dual lumen tubing 516. In a preferred embodiment, the cut section 550 extends from the proximal-most edge of the oblique distal end 526 by a distance of about 1 mm to about 10 mm; more preferably, about 5 mm.

As shown in FIG. 21A, the proximal end 518 of the dual lumen tubing 516 may be cut or shaved longitudinally to form a proximal cut portion 519 which is similar to the cut portion 550 formed at the distal end 520 of the single lumen tubing 506. As with the cut portion 550, the proximal cut portion 519 further facilitates insertion of the single lumen tubing 506 into the dual lumen tubing 516. The proximal cut portion 519 preferably extends from the proximal end 518 of the dual lumen tubing 516 by a distance of about 1 mm to about 10 mm; and more preferably, by a distance of about 5 mm. In another embodiment, the proximal cut portion 519 may have a length along the dual lumen tubing 516 such that a distal edge of the cut portion 519 is spaced from the proximal end 524 of the guidewire lumen 546 by a distance of about 1 mm to about 8 mm; more preferably, about 3 mm to about 4 mm.

As best illustrated in FIG. 21A, the aspiration lumen 544 ends in a distal aspiration mouth 522, which preferably defines an oblique opening relative to the longitudinal axis of the aspiration lumen 544. The oblique aspiration mouth 522 improves flow or evacuation rate efficiency, and facilitates the aspiration of larger particles, having various orientations within the vessel 16, which might otherwise resist passage through or become lodged within a blunt tip 458 or a tapered tip 410 (FIGS. 14B-14C). In one embodiment, the aspiration mouth 522 has a cross-sectional area of about 0.0083 square inches. In addition, the oblique angle of the aspiration mouth 522 prevents suction from occurring between the aspiration lumen 546 and the distal balloon 12. This substantially eliminates the risk of damaging the balloon 12 or the distal tip 526, as well as forcible movement of the balloon 12 while it is inflated within the vessel 16. Furthermore, the oblique aspiration mouth 522 provides a low profile distal end 526 which facilitates navigation of the catheter 500 within tortuous blood vessel networks and reduces the tendency of the distal end 526 to snag and get hung up on other objects which may be in the vessel 16 such as a stent.

As illustrated in FIG. 25, aspiration occurs through both the aspiration lumen 542 of the single lumen tubing 506 and the aspiration lumen 544 of the dual lumen tubing 516. The aspiration mouth 522 preferably has a length along the aspiration lumen 544 of about 6 mm. The width of the aspiration mouth 522 generally depends on the major diameter of the aspiration lumen 544, discussed with reference to FIG. 22, and preferably is about 0.050 inches. This cross-sectional shape facilitates extrusion.

After the single lumen tubing 506 is inserted into the dual lumen tubing 516, an adhesive is applied to hold the two pieces of tubing together. As shown in FIG. 21, a shrink tubing 548 formed of polyethylene terephthalate (PET) or other suitable material is provided over the junction of the single lumen tubing 506 and the dual lumen tubing 516. The shrink tubing 548 provides a mechanical bond which increases the strength of the junction while minimizing the cross-sectional profile thereof. In one embodiment, the cross-sectional profile of the junction of the single lumen tubing 506 and the dual lumen tubing 516 preferably is not more than about 0.069 inches. Furthermore, the shrink tubing 548 provides additional support to the proximal end 524 of the guidewire lumen 546, helping to keep the proximal end 524 open during aspiration procedures. The shrink tubing 548 preferably extends distally from the proximal end 524 of the guidewire lumen 546 by a distance of about 5 mm to about 30 mm, more preferably about 15 mm, and proximally from the proximal end 524 of the guidewire lumen 546 by a distance of about 5 mm to about 30 mm, more preferably about 15 mm. In another embodiment, the shrink tubing 548 extends proximally from the proximal end 524 of the guidewire lumen 546 to between about 0.5 mm to about 1.5 mm from the transition area 514; more preferably about 1.0 mm. The shrink tubing 548 preferably has a thickness such that the necked portion of the single lumen tubing 506, distal of the transition area 514, has a diameter of no more that about 0.054 inches.

In order to keep the guidewire lumen 546 open during fabrication, a wire mandrel similar to the rod 492 of FIG. 15 can be inserted into the guidewire lumen 546 while the single lumen tubing 506 is being inserted into the aspiration lumen 544. Furthermore, such a mandrel preferably extends out of the proximal end 524 of the guidewire lumen 546 while the shrink tubing 548 is being applied, thereby keeping the proximal end 524 of the guidewire lumen 546 exposed.

As will be appreciated by those skilled in the art, in both the aspiration catheters 200, 500, the single lumen tubing of the catheter must have sufficient structural integrity, or “stiffness,” to permit the catheter to be pushed through the vasculature to distal arterial locations without buckling or undesirable bending of the single lumen tubing. It is also desirable, however, for the dual lumen tubing to be fairly flexible near its distal end, so that the dual lumen tubing may be navigated through tortuous blood vessel networks. Thus, in one embodiment, the dual lumen tubing 516 of the aspiration catheter 500 may be made to have variable stiffness along its length, with the distal portion of the dual lumen tubing 516 being less flexible than the proximal portion of the dual lumen tubing 516. In another embodiment, the dual lumen tubing may be more flexible than the single lumen tubing. In still another embodiment, the dual lumen tubing 516 has a tensile strength of about 5,000 psi and the single lumen tubing 506 has a tensile strength of about 20,000 psi. A dual lumen tubing of this construction advantageously enables a physician to more easily insert the catheter into vascular networks that are otherwise difficult to access using conventional catheters of uniform stiffness. This is because the stiffer proximal portion provides the requisite structural integrity needed to advance the catheter without buckling, while the more flexible distal portion is more easily advanced into and through tortuous blood vessel passageways.

In one preferred embodiment, variable stiffness along the length of the dual lumen tubing of the catheter is achieved by forming a polymeric dual lumen tubing which incorporates a reinforcement along its length. For example, the dual lumen tubing may be provided with a reinforcing braid or coil incorporated into its wall structure. The reinforcement can be formed of metal or of various polymers. To achieve variable stiffness, the distal portion of the catheter is provided with a braid or coil having a higher braid or coil density than that present in the braid or coil of the proximal portion. The lower braid density in the proximal portion makes it less flexible, or “stiffer,” than the distal portion of the catheter.

The precise density of the braiding or coiling provided to the proximal, distal and transition portions can be varied considerably at the time of manufacture, such that catheters having a variety of different flexibility profiles may be created. Moreover, the braid or coil density may be varied within the catheter portions as well, by providing a braid or coil which has a braid or coil density gradient along its length. For example, the proximal-most part of the proximal portion may be provided with a metallic braid having a braid density of about 50-90 picks per inch, with the braid density increasing at a rate of about 2-5 picks per inch as the braid extends in the distal direction. This reinforced construction of the catheter provides adequate proximal stiffness for axial push, while preventing collapse of the distal tip during aspiration.

A variety of different materials, known to be ductile and shapeable into fine wires, may be used to form the reinforcement. For example, various polymers, stainless steel, silver or gold plated stainless steel, platinum, nitinol, or a combination thereof are suitable. In one embodiment, the braid is formed of stainless steel, and has a braid density which varies from 50-70 picks per inch at the most proximal part of the proximal region of the catheter, to 80-100 picks per inch at the most distal part of the distal region of the catheter.

Reinforcing braids or coils may be introduced into the structure of the dual lumen tubing through conventional catheter forming techniques. For example, the dual lumen tubing may be formed by inserting a 72D PEBAX tube into a variable braid density stainless steel sleeve, and then inserting the sleeved tube into a 72D PEBAX outer tube of the same length, so that the braided sleeve is sandwiched between the two tubes. A shaping mandrel may be inserted within the inner PEBAX tube, and shaping container over the outer PEBAX tube, and the entire apparatus may then be placed in a hot box kept at a temperature slightly greater than the melting temperature of the PEBAX tubes. The PEBAX tubes will melt and fuse together, and once cooled, will form a dual lumen tubing incorporating the braid. This same technique can be used to form a dual lumen tubing incorporating a coil.

In another embodiment, variable stiffness of the dual lumen tubing may be achieved by forming the proximal and distal portions of the dual lumen tubing out of polymeric materials having differing degrees of stiffness. For example, one half of an inner tube of 72D PEBAX may be inserted into an outer tube of 40D PEBAX, and the other half of the inner tube may be inserted into a 72D PEBAX outer tube. The combination may then be heat fused, as described above. The 40D/72D PEBAX combination forms a more flexible dual lumen tubing than the portion of the 72D/72D PEBAX combination. More or less flexible materials may be used as desired to alter the flexibility of the resulting dual lumen tubing. Furthermore, the flexibility of the various portions of a dual lumen tubing formed in this manner may be varied further by incorporating a braid or coil having either a uniform braid density or coil pitch, or a varying density or coil, into the dual lumen tubing, as described above.

Moreover, any of a variety of different polymeric materials known by those of skill in the art to be suitable for catheter body manufacture may be used to form the catheter body. For example, the body may be formed out of polymers such as polyethylene, PEBAX, polyimide, polyether etherketone, and the like. Different materials might also be combined to select for desirable flexibility properties.

Also, although the catheter body has been described in the context of having two portions of differing flexibility, it will be readily appreciated by those of skill in the art that three or more portions of differing flexibility may easily be provided, by adapting the teachings contained herein.

In one embodiment, the single lumen tubing 506 of the catheter 500 is preferably made of polyimide or PEEK, or a combination thereof. The dual lumen tubing 516 is preferably made of polyethylene. It will be appreciated that other materials may also be used, as discussed herein. The junction of the single and dual lumen tubing 506, 516 is preferably made as small as possible. In one embodiment, the junction of the single and dual lumen tubing 506, 516 has an outer diameter of no more than about 0.069 inches, making the catheter 500 particularly well suited for use with a 7 French guide having a 0.072-inch inner diameter. Further details regarding aspiration catheters are described in the above-mentioned U.S. Pat. No. 6,152,909.

In operation, before using the aspiration catheter 500, the physician uses the guidewire 14, as well as the rest of the occlusion system described herein, to position and inflate the balloon 12 at a location within the vessel 16 distal of an area within the vessel 16 requiring treatment. With the vessel 16 sufficiently occluded, the occlusion system is removed from the guidewire 14. The physician then delivers and exchanges one or more therapy catheters over the guidewire 14 to perform treatment on the vessel 16. The balloon 12 isolates any particles that are expelled into the vessel 16 due the treatment. Once the treatment is finished, the physician exchanges the therapy catheter with the aspiration catheter 500. Further details of this exchange are described in the above-mentioned application, entitled EXCHANGE METHOD FOR EMBOLI CONTAINMENT. With the therapy catheter removed, the physician inserts the proximal end of the guidewire 14 into the distal end 526 of the catheter 500. The guidewire 14 passes through the guidewire lumen 546 and exits through the proximal end 524 as the catheter 500 is advanced into the patient's vasculature.

As described above, the physician can rapidly advance the catheter 500 into the patient's vasculature, until the marker 530B is just outside the patient's body. At this point, the marker 530B indicates to the physician to slow the insertion of the catheter 500 and to turn on the fluoroscopy to carefully deliver the aspiration mouth 522 of the catheter 500 to the desired position. Using the fluoroscopy to observe the location of the marker 528 within the patient, the physician advances the aspiration mouth 522 to an optimal position proximal of the particles within the vessel 16. Preferably, the optimal position is about 8 mm to about 10 mm proximal of the particles to be aspirated. Upon applying a negative pressure to the aspiration lumen 544, the physician then aspirates the particles in the vessel 16. The physician may periodically advance and retract the catheter 500 to ensure that all of the particles are aspirated. Once the aspiration procedure is completed, the physician removes the source of negative pressure from the catheter 500 and then removes the catheter 500 from the patient's vasculature. The physician then reattaches the occlusion system, described with reference to FIGS. 1 through 4B, to the proximal end of the guidewire 14 and then deflates the balloon 12, thus restoring normal blood flow within the vessel 16. The guidewire 14 and the deflated balloon 12 are then removed from the patient.

One of the important features of the embodiments of the aspiration catheter is the ability, given sufficient irrigation fluid (which may be preferably the patient's own blood flowing in the vessel, or which may be other irrigation fluid supplied to the working area) to rapidly and efficiently aspirate even larger embolic particles without the need to first break them into smaller sub-particles. As discussed above, such embolic particles can comprise plaque or plaque pieces, thrombus, tissue, etc. This advantage is achieved, at least in part, through the relatively large size of the inner diameter (ID) of the aspiration lumen of the catheter, which in one embodiment is about 1 mm. Preferably, the ID of the aspiration lumen may fall within the range of 0.60-1.5 mm. This ID is more or less continuously maintained in the various embodiments of the aspiration catheter from proximal end to distal end, notwithstanding the junction between a single lumen catheter to a double or dual lumen catheter which accommodates a guide wire lumen. In those embodiments which have a crescent, or non-round, aspiration lumen configuration, an equivalent cross-sectional area is maintained to achieve rapid and efficient aspiration. Also, with these inner diameters, the outer diameter or cross-sectional profile can also be maintained at a minimal level in order to allow the catheter to traverse virtually all vessels in achieving aspiration. These inner diameters of the aspiration lumen, together with the large size of the aspiration opening or mouth, allows the catheter to aspirate larger particles, such as those on the order of 200-2500 microns. This has been shown to be possible in clinical trials.

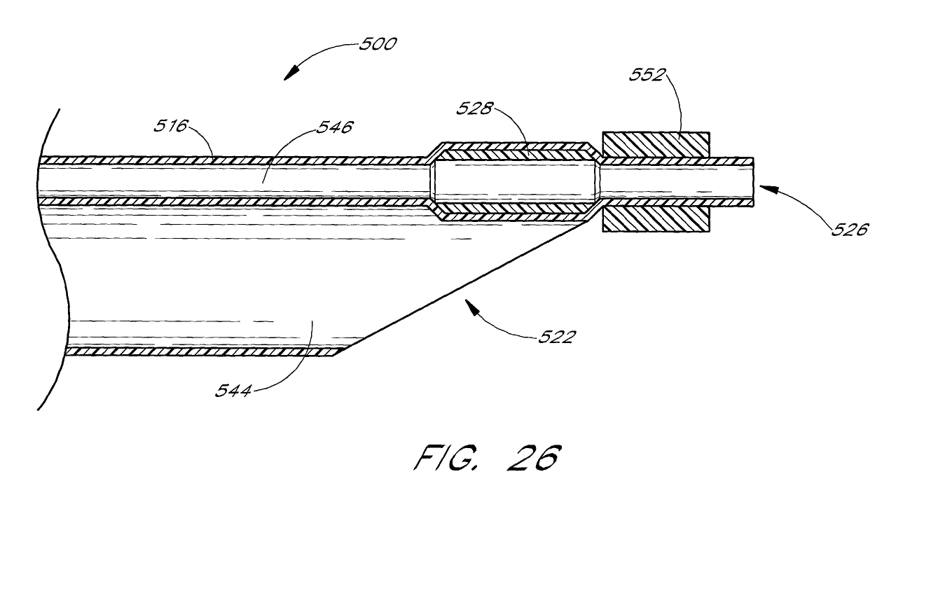

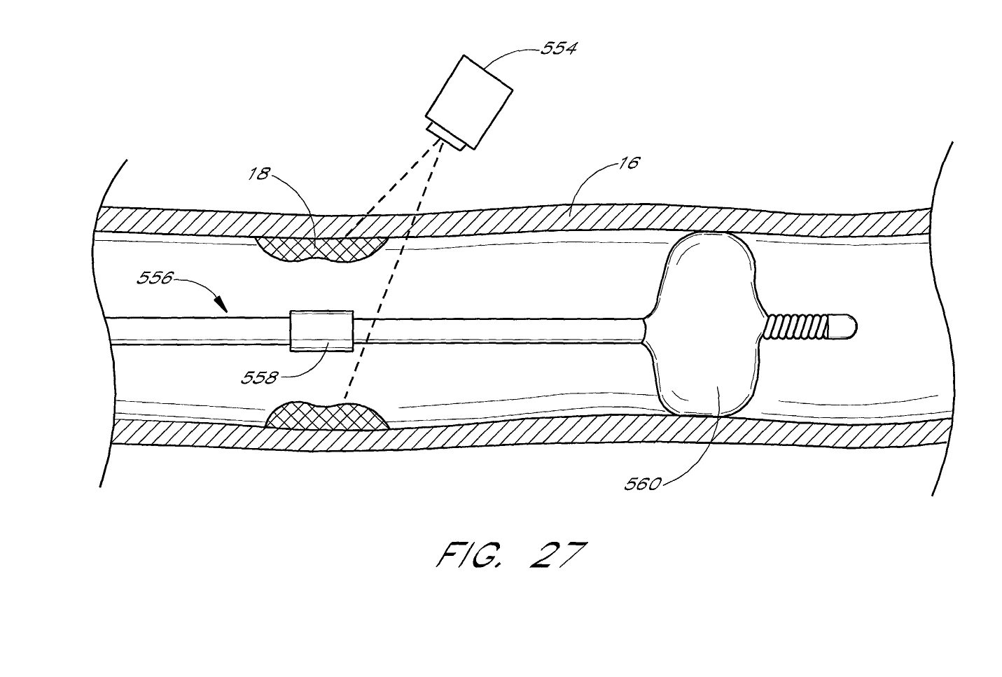

FIG. 26 illustrates one embodiment of an ultrasound sensor 552 positioned near the distal end 526 of the aspiration catheter 500. The sensor 552 is preferably located near the marker 528, and more preferably is located just distal of the marker 528. It will be appreciated, however, that the sensor 552 may be placed anywhere near the aspiration mouth 522, proximal or distal of the marker 528 and positioned over just the guidewire lumen 546 or both the guidewire and aspiration lumens 546, 544. The advantage of placing the ultrasound sensor 552 on the aspiration catheter 500 is that after aspiration is completed, this same catheter 500 can be used to focus ultrasonic shockwaves produced by a shock wave generator 554 (FIG. 27) to determine whether all or at least a substantial number of particles have been successfully aspirated. If a substantial number of particles remains, further aspiration can immediately be applied to remove the additional particles because the aspiration catheter 500 remains in the vessel 16. It should be noted that although FIG. 26 shows the ultrasound sensor 552 utilized with the aspiration catheter 500, the ultrasound sensor 552 can also be utilized with the aspiration catheter 200, illustrated in FIG. 17, in the manner described above.

FIG. 27 shows an embodiment of a distal occlusion catheter 556 for use in directing ultrasonic shockwaves to disintegrate plaque 18 within a vessel 16. The catheter 556 comprises a radiopaque marker 558 located proximal of a distal balloon 560. The marker 558 is used to locate the plaque 18 for targeting by the external shock wave generator 554. After inflation of the balloon 560, the shock wave generator 554 is focused onto the plaque 18 by use of the radiopaque marker 558 to disintegrate the plaque 18.

After treatment of the plaque 18 by the shock wave generator 554, an aspiration catheter may be passed over the guidewire 556 for aspirating the emboli created by the shock wave treatment. Alternatively, the shock wave treatment may be performed with the aspiration catheter already advanced over the guidewire 556. In such an embodiment, the radiopaque marker 558 may either be placed on the guidewire 556 or the aspiration catheter itself for targeting the location of the plaque 18.

Unless otherwise noted, the method steps described herein can be performed in any desired order and are not intended to be construed as necessarily being performed sequentially.

Various embodiments have been described above. Although these embodiments have been described with reference to specific materials and configurations, the descriptions are intended to be illustrative only and are not intended to be limiting. It will be appreciated that the specific dimensions of the various catheters and guidewires can differ from those described above, and that the methods described can be used within any biological conduit within the body. Various modifications and applications may occur to those skilled in the art without departing from the scope of the invention as defined in the appended claims.

What is claimed is:

1. An aspiration catheter for removing emboli or other particles from a blood vessel, comprising:
a first elongate body having a proximal end and a distal end and a first lumen extending therethrough; and
a second elongate body having a proximal end and a distal end and a second lumen and a third lumen extending therethrough;
wherein said first lumen of said first elongate body is inserted into said second lumen of said second elongate body to form an aspiration lumen extending from said proximal end of said first lumen to said distal end of said second lumen, and wherein said third lumen extends substantially parallel to said second lumen and has a proximal end proximal to said distal end of said first lumen and distal to said proximal end of said second lumen, and a distal end distal to said distal end of said second lumen, and wherein said third lumen is adapted to receive a guidewire therethrough.

2. The aspiration catheter of claim 1, wherein said third lumen extends within the second lumen.

3. The aspiration catheter of claim 1, wherein said second elongate body has a length between about 35 centimeters and about 36 centimeters.

4. The aspiration catheter of claim 3, wherein said length is about 35 centimeters.

5. The aspiration catheter of claim 1, wherein said aspiration catheter has a length between about 145 centimeters and about 150 centimeters.

6. The aspiration catheter of claim 1, wherein a distal portion of said first elongate body has a first diameter and a remaining proximal portion of said first elongate body has a second diameter, said first diameter being less than said second diameter.

7. The aspiration catheter of claim 6, wherein said first diameter is about 0.040 inches to about 0.042 inches.

8. The aspiration catheter of claim 7, wherein said first diameter is about 0.041 inches.

9. The aspiration catheter of claim 7, wherein said second diameter is about 0.052 inches.

10. The aspiration catheter of claim 9, wherein said first elongate body includes a plurality of markers each having a thickness such that said second diameter is no more than about 0.054 inches.

11. The aspiration catheter of claim 10, wherein a first of said plurality of markers is spaced about 43 centimeters from said distal end of said third lumen.

12. The aspiration catheter of claim 10, wherein a second of said plurality of markers is spaced about 90 centimeters from said distal end of said third lumen.

13. The aspiration catheter of claim 1, wherein said second elongate body has an outside diameter of about 0.060 inches.

14. The aspiration catheter of claim 1, wherein said distal end of said second lumen comprises an aspiration mouth having an oblique angle relative to a longitudinal axis of said second lumen, said aspiration mouth facing away from said third lumen and being in fluid communication with said second lumen.

15. The aspiration catheter of claim 14, wherein said aspiration mouth has a cross-sectional area of about 0.0083 square inches.

16. The aspiration catheter of claim 14, wherein said aspiration mouth has a length along said longitudinal axis of about 4 mm to about 8 mm.

17. The aspiration catheter of claim 16, wherein said length is about 6 mm.

18. The aspiration catheter of claim 14, wherein said distal end of said third lumen extends beyond said aspiration mouth by a distance of about 0.5 mm to about 5 mm.

19. The aspiration catheter of claim 18, wherein said distance is about 1.5 mm.

20. The aspiration catheter of claim 1, wherein said second lumen has a cross-sectional area of about 0.0018 square inches.

21. The aspiration catheter of claim 1, wherein said aspiration catheter provides an evacuation flow rate of about 0.5 cc/second to about 0.9 cc/second.

22. The aspiration catheter of claim 21, wherein said aspiration catheter provides an average evacuation flow rate of about 0.7 cc/second.

23. The aspiration catheter of claim 21, wherein said aspiration catheter provides an optimal evacuation flow rate of at least about 0.68 cc/second.

24. The aspiration catheter of claim 1, wherein said distal end of said third lumen has a maximum outside diameter of no more than about 0.025 inches.

25. The aspiration catheter of claim 1, wherein said proximal end of said first elongate body is fitted with an aspiration port in fluid communication with said second lumen.

26. The aspiration catheter of claim 25, wherein said aspiration port is configured to receive a source of negative pressure.

27. The aspiration catheter of claim 1, wherein said second elongate body comprises a cut section extending distally from said proximal end of said second lumen to a distance proximal of said proximal end of said third lumen.

28. The aspiration catheter of claim 27, wherein said distance is about 1 mm to about 8 mm.

29. The aspiration catheter of claim 28, wherein said distance is about 3 mm to about 4 mm.

30. The aspiration catheter of claim 1, wherein said distal end of said first elongate body comprises a cut section extending proximally from said distal end by a distance which directly corresponds with a length of said first elongate body which is inserted into said second lumen.

31. The aspiration catheter of claim 1, wherein said distal end of said first elongate body has an oblique angle relative to a longitudinal axis of said first elongate body.

32. The aspiration catheter of claim 31, wherein said oblique angle is between about 10 degrees and about 45 degrees.

33. The aspiration catheter of claim 31, wherein said oblique angle is about 30 degrees.

34. The aspiration catheter of claim 1, wherein an adhesive is used to affix said distal end of said first lumen to said proximal end of said second lumen.

35. The aspiration catheter of claim 1, wherein said first lumen is secured to said second lumen by a length of shrink tubing which is contracted around an interface between said first and second lumens.

36. The aspiration catheter of claim 35, wherein said shrink tubing is formed of polyethylene terephthalate.

37. The aspiration catheter of claim 35, wherein said shrink tubing extends distally from said proximal end of said third lumen by a distance of about 5 mm to about 30 mm.

38. The aspiration catheter of claim 37, wherein said distance is about 15 mm.

39. The aspiration catheter of claim 35, wherein said shrink tubing extends proximally from said proximal end of said third lumen to between about 0.5 mm to about 1.5 mm from a transition area of said first elongate body, said transition area comprising transition from a reduced diameter to a larger diameter of said first elongate body.

40. The aspiration catheter of claim 39, wherein said shrink tubing extends proximally from said proximal end of said third lumen to about 1.0 mm from said transition area.

41. The aspiration catheter of claim 35, wherein said interface and said shrink tubing provide a maximal diameter of said second elongate body of no more than about 0.069 inches.

42. A method of fabricating an aspiration catheter for removing emboli or other particles from a blood vessel, comprising:
providing a first elongate tubular body having a single lumen extending therethrough;
affixing an aspiration port to a proximal end of said first elongate tubular body such that said aspiration port is in fluid communication with said single lumen;
heating and stretching a distal portion of said first elongate tubular body to narrow the diameter of said distal portion;
removing material from one side of a distal end of said first elongate tubular body to form a cut section;
providing a second elongate tubular body having a primary lumen extending therethrough and a secondary lumen extending within said primary such that said primary lumen has a crescent cross-section and said second elongate tubular body has a round cross-section, said secondary lumen being substantially parallel with said primary lumen, said secondary lumen extending distally beyond an aspiration mouth of said primary lumen and forming a distal end of said aspiration catheter, said aspiration mouth having an oblique angle relative to a longitudinal axis of said primary lumen and being in fluid communication therewith, said aspiration mouth facing away from said secondary lumen;
inserting a rod into said distal end of said secondary lumen such that a distal end of said rod is outside of said distal end of said secondary lumen and a proximal end of said rod protrudes from a proximal end of said secondary lumen;
forming a junction by inserting said distal end of said first elongate tubular body into a proximal end of said primary lumen such that said cut section faces towards said secondary lumen; and
positioning a length of shrink tubing over said junction and causing said shrink tubing to contract thereon.

43. The method of claim 42, further comprising applying an adhesive to said junction.

44. The method of claim 42, wherein said cut section extends from said distal end of said first elongate tubular body by a distance which directly corresponds with a length of said first elongate tubular body which is inserted into said primary lumen.

45. The method of claim 42, wherein said rod comprises a distal ball attached to an elongate shaft.

46. The method of claim 45, wherein said rod is a wire mandrel.

47. The method of claim 42, wherein said shrink tubing is formed of polyethylene terephthalate.

48. The method of claim 42, wherein said shrink tubing extends distally from said proximal end of said secondary lumen by a distance of about 5 mm to about 30 mm.

49. The method of claim 48, wherein said distance is about 15 mm.

50. The method of claim 42, wherein said shrink tubing extends proximally from said proximal end of said secondary lumen to about 0.5 mm to about 1.5 mm from a transition area of said first elongate tubular body, said transition area comprising a proximal end of said distal portion of said first elongate tubular body.

51. The method of claim 50, wherein said shrink tubing extends proximally from said proximal end of said secondary lumen to about 1.0 mm from said transition area.

52. The method of claim 42, wherein said primary lumen has a cross-sectional area of about 0.0018 square inches.

53. The method of claim 42, wherein said aspiration mouth has a cross-sectional area of about 0.0083 square inches.

54. The method of claim 42, further comprising inserting a marker within said distal end of said secondary lumen, said inserting further comprising positioning said marker within said secondary lumen at the position of said aspiration mouth.

55. The method of claim 42, wherein said aspiration catheter provides an evacuation flow rate of about 0.5 cc/second to about 0.9 cc/second.

56. The method of claim 55, wherein said aspiration catheter provides an average evacuation flow rate of about 0.7 cc/second.

57. The method of claim 55, wherein said aspiration catheter provides an optimal evacuation flow rate of at least about 0.68 cc/second.

58. An aspiration catheter for removing emboli or other particles from a blood vessel, comprising:
a proximal portion having a first lumen extending therethrough;
a distal portion having a second lumen and a third lumen extending therethrough, said third lumen extending within said second lumen and being substantially parallel therewith such that said second lumen has a crescent cross-section and said distal portion has a round cross-section; and
a junction comprising said proximal portion being distally inserted into a proximal end of said distal portion such that said first lumen is in fluid communication with said second lumen.

59. The aspiration catheter of claim 58, wherein an adhesive is used to affix said proximal portion to said distal portion.

60. The aspiration catheter of claim 58, wherein said proximal portion is secured to said distal portion by a length of shrink tubing which is contracted around said junction.

61. The aspiration catheter of claim 60, wherein said shrink tubing is formed of polyethylene terephthalate.

62. The aspiration catheter of claim 58, wherein a distal end of said proximal portion comprises a cut section extending proximally from said distal end, said cut section having a length directly proportional to a length of said proximal portion which is inserted into said distal portion.

63. The aspiration catheter of claim 58, wherein said third lumen comprises a proximal end which is distal of said proximal end of said distal portion.

64. The aspiration catheter of claim 63, wherein said distal portion comprises a cut section extending distally from a proximal end of said second lumen to a distance proximal of said proximal end of said third lumen.

65. The aspiration catheter of claim 58, wherein said second lumen has a cross-sectional area of about 0.0018 square inches.

66. The aspiration catheter of claim 58, wherein a distal end of said second lumen comprises an aspiration mouth having an oblique angle relative to a longitudinal axis of said second lumen, said aspiration mouth facing away from said third lumen and being in fluid communication with said second lumen.

67. The aspiration catheter of claim 66, wherein said aspiration mouth has a cross-sectional area of about 0.0083 square inches.

68. The aspiration catheter of claim 65, wherein said aspiration mouth has a length along said longitudinal axis of about 4 mm to about 8 mm.

69. The aspiration catheter of claim 68, wherein said length is about 6 mm.

70. The aspiration catheter of claim 65, wherein said third lumen extends beyond said aspiration mouth by a distance of about 0.5 mm to about 5 mm.

71. The aspiration catheter of claim 70, wherein said distance is about 1.5 mm.

72. The aspiration catheter of claim 58, wherein said aspiration catheter provides an evacuation flow rate of about 0.5 cc/second to about 0.9 cc/second.

73. The aspiration catheter of claim 72, wherein said aspiration catheter provides an average evacuation flow rate of about 0.7 cc/second.

74. The aspiration catheter of claim 72, wherein said aspiration catheter provides an optimal evacuation flow rate of at least about 0.68 cc/second.

75. The aspiration catheter of claim 58, wherein said third lumen is sized and configured to receive a guidewire.

76. The aspiration catheter of claim 58, wherein a proximal end of said proximal portion is fitted with an aspiration port in fluid communication with said second lumen.

77. The aspiration catheter of claim 76, wherein said aspiration port is configured to receive a source of negative pressure.

78. An aspiration catheter for removing emboli or other particles from a blood vessel, comprising:
a dual lumen portion having a primary lumen and a secondary lumen, said primary lumen having a distal aspiration mouth in fluid communication with said primary lumen, said secondary lumen extending within said primary lumen and protruding distally beyond said aspiration mouth to form a distal end of said aspiration catheter;
a single lumen portion having a distal end inserted into a proximal end of said primary lumen such that a proximal end of said single lumen portion is in fluid communication with said aspiration mouth; and
an aspiration port disposed on said proximal end of said single lumen portion and in fluid communication with said aspiration mouth.

79. The aspiration catheter of claim 78, wherein said secondary lumen is substantially parallel with said primary lumen such that said primary lumen has a crescent cross-section and said dual lumen portion has a round cross-section.

80. The aspiration catheter of claim 78, wherein said aspiration port receives a source of negative pressure.

81. The aspiration catheter of claim 78, wherein said primary lumen has a cross-sectional area of about 0.0018 square inches.

82. The aspiration catheter of claim 78, wherein said aspiration mouth defines an oblique opening facing away from said secondary lumen.

83. The aspiration catheter of claim 82, wherein said aspiration mouth has a cross-sectional area of about 0.0083 square inches.

84. The aspiration catheter of claim 78, wherein said aspiration catheter provides an evacuation flow rate of about 0.5 cc/second to about 0.9 cc/second.

85. The aspiration catheter of claim 84, wherein said aspiration catheter provides an average evacuation flow rate of about 0.7 cc/second.

86. The aspiration catheter of claim 84, wherein said aspiration catheter provides an optimal evacuation flow rate of at least about 0.68 cc/second.

87. The aspiration catheter of claim 78, wherein an adhesive is used to affix said single lumen portion to said dual lumen portion.

88. The aspiration catheter of claim 78, wherein said single lumen portion is secured to said dual lumen portion by a length of shrink tubing which is contracted around an interface between said single lumen portion and said dual lumen portion.

89. The aspiration catheter of claim 88, wherein said shrink tubing is formed of polyethylene terephthalate.

90. The aspiration catheter of claim 78, wherein said secondary lumen is sized and configured to receive a standard-size coronary guidewire.

91. The aspiration catheter of claim 78, wherein said distal end of said single lumen portion comprises a cut section extending proximally from said distal end, said cut section having a length which is directly proportional to a length of said single lumen portion which is inserted into said dual lumen portion.

92. The aspiration catheter of claim 78, wherein a proximal end of said secondary lumen is distal of said proximal end of said primary lumen.

93. The aspiration catheter of claim 92, wherein said dual lumen portion comprises a cut section extending distally from said proximal end of said primary lumen to a distance proximal of said proximal end of said secondary lumen.

94. A method of fabricating an aspiration catheter for removing emboli or other particles from a blood vessel, comprising:
providing a first elongate tubular body having a single lumen extending from a distal end to a proximal end, said proximal end being fitted with an aspiration port in fluid communication with said single lumen, said distal end having an oblique angle relative to a longitudinal axis of said first elongate tubular body, said distal end having a cut section extending proximally on one side;
providing a second elongate tubular body having a primary lumen extending therethrough and a secondary lumen extending within said primary lumen such that said primary lumen has a crescent cross-section and said second elongate tubular body has a round cross-section, said secondary lumen being substantially parallel with said primary lumen;
inserting said distal end of said first elongate tubular body into a proximal end of said primary lumen with said cut section facing towards said secondary lumen; and
securing said distal end of said first elongate tubular body to said proximal end of said primary lumen.

95. The method of claim 94, wherein said securing comprises using an adhesive.

96. The method of claim 94, wherein said securing comprises positioning a length of shrink tubing over said junction and causing said shrink tubing to contract thereon.

97. The method of claim 94, wherein said shrink tubing is formed of polyethylene terephthalate.

98. The method of claim 94, wherein said cut section extends from said distal end of said first elongate tubular body by a distance which directly corresponds with a length of said first elongate tubular body which is inserted into said primary lumen.

99. The method of claim 94, further comprising inserting a marker within said distal end of said secondary lumen, said inserting further comprising positioning said marker within said secondary lumen at the position of said aspiration mouth.

100. An aspiration catheter for removing emboli or other particles from a blood vessel, comprising:
a shaft comprising a distal end and a proximal end and having at least a first lumen and a second lumen extending therebetween, said second lumen extending within said first lumen such that said first lumen has a crescent cross-section and said shaft has a round cross-section;
an aspiration port disposed on said proximal end and being in fluid communication with said first lumen;
an aspiration mouth disposed on said distal end and being in fluid communication with said first lumen, said aspiration mouth defining an oblique opening which faces away from said second lumen; and
an opening disposed between said distal end and said proximal end of said shaft, said opening defining a proximal end of said second lumen and being in fluid communication with a distal end of said second lumen.
